(19) United States
(12) Patent Application Publication
(10) Pub. No.: US 2008/0025959 A1
(43) Pub. Date: Jan. 31, 2008

(54) Permeability of blood-brain barrier

(76) Inventors: Richard Daneman, Menlo Park, CA (US); Ben A. Barres, Palo Alto, CA (US)

Correspondence Address: WILSON SONSINI GOODRICH & ROSATI, 650 PAGE MILL ROAD, PALO ALTO, CA, 94304-1050, US

(21) Appl. No.: 11/807,245

(22) Filed: May 24, 2007

Related U.S. Application Data
(60) Provisional application No. 60/808,323, filed on May 24, 2006.

(51) Int. Cl. A61K 38/00; A61K 31/715; A61K 38/43; A61P 25/00

(52) U.S. Cl. 424/94.1; 514/2; 514/44

(57) ABSTRACT

This relates to a discovery of a cell surface protein, namely NgR2, that is implicated in regulation of blood-brain barrier (BBB). In addition, the invention relates methods for identifying agents that modulate BBB permeability. Furthermore, the invention relates to methods of delivering therapeutic agents to the central nervous system by increasing BBB permeability.

### CROSS REFERENCE

This application claims the benefit of U.S. Provisional Application No. 60/808,323, filed on May 24, 2006, which is incorporated herein by reference in its entirety.

### FEDERALLY SPONSORED WORK

The work in this application was partly funded by one or more grants by the National Institutes of Health NIH1 RO1 NS045621-01. The government has certain rights in this invention.

### BACKGROUND OF THE INVENTION

All organisms with complex nervous systems have a blood-brain barrier (BBB) to isolate the CNS from the blood. This barrier is important to maintain brain homeostasis as well as prevent the entry of toxic molecules, pathogenic organisms and the bodies own immune system from entering the brain. Understanding how the BBB is regulated has implications for understanding and developing treatments of a wide variety of neurological diseases.

Patients suffering from edema, brain traumas, stroke and multiple sclerosis exhibit a breakdown of the BBB near the site of primary insults. The level of breakdown can have profound effects on the clinical outcome of these diseases. For instance, the degree BBB breakdown in patients suffering from multiple sclerosis (MS) is correlated to the severity of the disease. It has been shown using Magnetic Resonance Imaging (MRI) that, when a person is undergoing an MS “attack,” the blood-brain barrier has broken down in a section of the brain or spinal cord, allowing white blood cells called T lymphocytes to cross over and destroy the myelin.

The BBB is also as an obstacle for the treatment of all CNS diseases by inhibiting the delivery of drugs to the brain. A major challenge for treatment of many CNS disorders or conditions is overcoming the difficulty of delivering therapeutic agents to specific regions of the brain. In its neuroprotective role, the blood-brain barrier functions to hinder the delivery of many potentially important diagnostic and therapeutic agents to the brain. Therapeutic molecules and genes that might otherwise be effective in diagnosis or therapy do not cross the BBB in adequate amounts. For example, chemotherapy has been relatively ineffective in the treatment of CNS metastases of systemic cancers including breast cancer, small cell lung cancer, lymphoma, and germ cell tumors, despite clinical regression and even complete remission of these tumors in non-CNS systemic locations. Many currently-available chemotherapeutic agents have a molecular weight larger than a typical cutoff size for BBB-penetratable molecules, or exhibit water or lipid solubility incompatible to BBB permeability.

The BBB can be differentiated from the peripheral tissue endothelia, as it possesses uniquely distinguishing structural characteristics. Cerebral capillary endothelial cells contain tight junctions, which seal cell-to-cell contacts between adjacent endothelial cells forming a continuous blood vessel. The tight junctions between BBB endothelial cells leads to high endothelial electrical resistance, in the range of 1500-2000 Ω·cm² (pial vessels), as compared to 3-33 Ω·cm² in other tissues. The electrical resistance across in vivo cerebral microvessel endothelial cells, of non-pial origin, has been estimated to be as high as 8000 Ω·cm². The net result of this elevated resistance is low paracellular permeability. Additional structural characteristics for the BBB include the periendothelial accessory structures such as pericytes, astrocytes, and a basal membrane. The endothelial cells of the BBB are distributed along the vessels and completely encircle the lumens. A thin basement membrane (i.e. basal lamina) supports the ablumenal surface of the endothelium. The basal lamina surrounds the endothelial cells and pericytes; the region between which is known as the Virchow-Robin space. Astrocytes are typically located adjacent to the endothelial cell, with astrocytic end feet sharing the basal lamina.

Evidence suggests that the physiological characteristics of the BBB are associated with the expression of a distinctive set of genes within the capillary endothelium and/or possibly cofactors from the surrounding tissue. Key transplantation studies have demonstrated that these properties are not exclusively intrinsic to the endothelial cells. Gut tissue vascularized by brain endothelial cells produce leaky vessels, while brain tissue vascularized by gut endothelial cells generate impermeable vessels reminiscent of the BBB. This observation has led to the proposal that neural tissue regulates the formation of this crucial barrier. Much work has concentrated on the role of astrocytes in regulating the BBB, due to the fact that these glial cells send out processes which ensheath vessels. While the exact role of astrocytes remains controversial, several experiments suggest these cells play a vital role regulating the BBB. Transplantation of purified astrocytes was sufficient to reduce the permeability of vessels in the chick chorioallantoic membrane as well as the anterior chamber of the rat eye. Furthermore, co-culture of astrocytes with purified endothelial cells in vitro elevates the electrical resistance of the endothelial monolayers. However, other work suggests that the BBB re-forms after injury prior to astrocyte ensheathment.

One of the previously uncharacterized rat brain antigens is endothelial brain antigen (EBA) which is recognized by a commercially available antibody (SMI71). EBA has been shown to be present in vessels of the optic nerve, retina, iris, and spinal cord, as well as vessels of pia mater. However, EBA is absent from endothelial cells that lack barrier properties of body organs, such as intestine, kidney, liver, thyroid and pancreas, none of which possess a blood-tissue barrier, or express the EBA antigen.

In addition, one study has demonstrated that intravenously injected anti-EBA antibody binds ECs, detectable in tissue sections and effected disruption of the BBB, which disruption was not observed in animals administered a control antibody (Ghabriel et al. Brain Research. 2000; 878:127-35). While it has been described that EBA can be targeted to disrupt BBB permeability, the particular target antigen has not been identified.

Despite the importance of this barrier, very little is known about the molecular mechanisms controlling the integrity and/or permeability of the BBB. Research for the underlying molecular mechanisms has been hampered by the continuing lack of model systems, and especially a reversible model system whose BBB permeability can be exogenously manipulated. Thus, there remains a considerable need for compositions and methods that facilitate such research and especially for diagnostic and/or therapeutic applications. The present invention addresses these needs and provides related advantages as well.

### SUMMARY OF THE INVENTION

One aspect of the present invention is the discovery that the NgRH1 cell surface receptor, an antigen preferentially expressed in endothelial cells, is involved in regulating blood-brain barrier (BBB) permeability. Accordingly, the present invention provides a method of modulating BBB permeability comprising the step of administering an agent to a subject, wherein said agent targets a human NgRH1 cell surface receptor that is present in the brain. In a related but separate embodiment, the BBB permeability modulation method involves administering a ligand of NgRH1 cell surface receptor protein to the subject. In one aspect, the agent administered is characterized by its ability to increase BBB permeability. Such modulation is preferably reversible or transient, so as to avoid permanent damages to the CNS. In one aspect, the agent administered is characterized by its ability to decrease BBB permeability. Non-limiting examples of the agents useful for modulating BBB permeability via NgRH1 includes inorganic molecules, peptides, peptide mimetics, antibodies, liposomes, small interfering RNAs, antisense, aptamers, and external guide sequences.

The present invention also provides a method of delivering a therapeutic agent to a central nervous system (CNS) of a subject. The method typically involves the step of administering the therapeutic agent to the CNS prior to, concurrent with, or subsequent to, increasing BBB permeability as a result of modulating NgRH1 cell surface receptor activity and/or expression level. In one aspect, the NgRH1 receptor is a human NgRH1.

The present invention further provides a method of assessing whether a candidate agent can modulate BBB permeability. This method generally comprises the step (a) exposing a subject's central nervous system to an indicator of said BBB permeability; (b) administering to said subject said candidate agent that targets NgRH1 cell surface receptor, wherein an increases or decrease in BBB permeability indicates that said candidate agent is capable of modulating BBB permeability. Where desired, the agent tested may be an agonist or an antagonist of NgRH1 cell surface receptor (including a splice variant thereof). Such method can be performed in an animal model including an transgenic animal.

Also provided in the present invention is a non-human transgenic animal comprising a genetic modification in NgRH1. The modification may results in a decreased or increased expression of NgRH1 in the CNS of the animal.

Other methods for treating CNS conditions, BBB-related disorders and conditions exhibiting a clinical manifestation in the CNS are provided herein as well.

### BRIEF DESCRIPTION OF THE DRAWINGS

FIG. 1. Provides an illustration of the structural characteristics of peripheral versus brain capillaries.

FIG. 2. Illustrates visualization of the blood-brain barrier. An anesthetized adult rat was cardiac perfused with the molecular tracer biotin. Fixed tissues were then sectioned and stained with a streptavidin alexa-488. Biotin steptavidin complexes were visualized by fluorescent microscopy. The biotin tracer stays within the lumen of the capillaries coursing through the brain tissue, while in contrast, in the muscle the tracer leaves the vessels and is able to diffuse throughout the extracellular space.

FIG. 3. Provides an illustration of cellular interactions involved in BBB development.

FIG. 4. Provides an illustration of the cell biology of capillaries.

FIG. 5. Provides an illustration of the structural characteristics of an optic nerve.

FIG. 6. Illustrates immunostaining of endothelial cell tight junctions. In the left panel, the cross-section of a blood vessel in an adult rat brain was co-labeled with occludin (red) and BSL (green). The tight junctions can be identified by occludin staining, joining neighboring endothelial cells. In the right panel is a schematic representation of a tight junction (Gloor et al. Brain Res Brain Res Rev. 2001 October; 36(2-3):258-64).

FIG. 7. Illustrates claudin immunostaining of occludin and claudin 5. Occludin is specific to the blood-brain barrier. Rat optic nerve and spleen tissues were stained with antibodies directed against occludin and claudin 5. Claudin 5 (right panels) is expressed by endothelial cells in both tissues, while occludin (left panels) is specific to endothelial cells in the CNS.

FIG. 8. Illustrates immunostaining of tight junctions. Time course of Occludin and Claudin 5 expression in the optic nerve. Claudin 5 (top panels) is expressed in the optic nerve endothelial cells as soon as they are generated at embryonic day 19.5. Meanwhile, occludin expression is delayed and is observed after birth.

FIG. 9. Illustrates that blood-brain barrier maturity proceeds after birth. Rhodamine-conjugated dextrans were transcardiacally perfused into Sprague dawley rats. In mature animals (right panel) the tracer stays within the lumen of the capillaries that course throughout the optic nerve demarcating a functional blood-brain barrier. Meanwhile, at early postnatal time points (left panel) the tracer is able to diffuse throughout the optic nerve, suggesting the blood-brain barrier is not fully mature.

FIG. 10. Illustrates a time course for BBB development. Full barrier is observed around P7 to P10.

FIG. 11. Illustrates immunostaining of pericytes and endothelial cells. Purification of vascular cells from the optic nerve. Rat optic nerves were enzymatically dissociated with papain, followed by negative immunopanning with C5 antibody. Endothelial cells were selected using an antibody directed against CD31, while pericytes were selected using an antibody against PDGFR beta.

FIG. 12. Illustrates occludin staining. Pericytes induce occludin expression in optic nerve endothelial cells. Rat optic nerve endothelial cells were cultured alone (top panel) or with optic nerve pericytes (lower panel), optic nerve astrocytes or retinal ganglion cells (data not shown). Occludin expression was monitored by immunofluoresence. Endothelial cells expressed occludin at cell borders when co-cultured with pericytes, but not alone, with astroyctes or neurons.

FIG. 13. Illustrates occludin staining. Endothelial cells were cultured in growth medium (top panel) or with medium conditioned by a layer of optic nerve endothelial cells (lower panel) or optic nerve astrocytes (data not shown). Occludin expression was only observed when pericyte conditioned medium was added.

FIG. 14. Illustrates optic nerve staining. Tissue structure of a rat eyeball. Frozen tissue sections of a rat eyeball were stained with the nuclear dye DAPI. The anterior chamber of the eye is formed betweend the lens, iris cilliary muscle and cornea.

FIG. 15. Illustrates immunostaining of vascularized transplanted cells. Purified astrocytes (left panel) or astrocytes with optic nerve pericytes (right panel) were transplanted into the anterior chamber of a rat eye. Transplants were visualized by staining with GFAP (green) and could be identified on the iris (left panel) ciliary muscle (right panel) or cornea (data not shown). In each case vasculature was identified next to each transplant by immunostaining for claudin 5 (red).

FIG. 16. Vasculature of pericyte transplants expressed occludin. Vasculature of transplanted astrocytes (left panel) or astrocytes with optic nerve pericytes (right panel) were immunostained with GFAP (green) to identify transplanted tissue, and occludin (red). Occludin was expressed in adjacent tissue when pericytes/astrocyte (right panel) mixtures were transplanted, but not astrocytes alone (left panel).

FIG. 17. Illustrates BBB disruption following systemic injection of SMI71 (anti-EBA antibody) is able to disrupt the blood-brain barrier. Sprague dawley rats were injected with 40 ul/kg SMI71 antibody (right panel) or CD31 antibody (left panel) and perfused with biotin 15 minutes after injection. Biotin tracer was detected in frozen brain tissue sections by fluorescent microscopy after staining with a streptavidin-alexa-488 (green). Animals injected with SMI71 (right panel) showed leakage of tracers from the capillaries into the brain parenchyma, while the tracer stayed within the lumen of the capillaries in CD31 injected animals (left panel).

FIG. 18. Illustrates expression cloning EBA antigen and selection of positive clones. An adult rat brain expression library (biochain) was divided into pools of 2000-4000 clones. Pools were transfected into COS-1 cells and screened by SMI71 binding with immunofluorescent microscopy. A single positive pool was identified (top left panel). Using a sib selection protocol, the number of clones per pool was narrowed down (top right, bottom right) until a positive pool with a single clone was identified. After sequencing this was identified as Ngrh1.

FIG. 19. Illustrates splice variant expression in endothelial cells. Ngrh1 mRNA is expressed by brain endothelial cells. RT-PCR was performed on RNA isolated from brain lysates (left lane), or CD31 purified endothelial cells from the spleen (right lane) or brain (second from the right lane) using primers specific to Ngrh1. Ngrh1 is expressed in all tissues. However, a specific splice variant is identified in brain endothelial cells but not spleen endothelial cells.

FIG. 20. Illustrates rat Nogo-66 receptor homolog-1 (Ngrh1) mRNA, complete coding sequence (SEQ ID NO: 9).

FIG. 21. Illustrates mouse Nogo-66 receptor homolog-1 (Ngrh1) mRNA, complete coding sequence (SEQ ID NO: 10).

FIG. 22. Provides a nucleic acid sequence encoding human NgRH1 (SEQ ID NO: 11).

FIG. 23. Illustrates spatial and temporal expression of EBA in the brain; A) Adult brain; B) P1 brain; C) P8 brain; D) P25 brain; E) P60 brain; F) & G) illustrate that SMI71 binds specifically to endothelial cells in acute dissociated cortical cultures; F) VWF; G) live SMI71.

FIG. 24. Illustrates breakdown of BBB after systemic injection of anti-EBA: A) α-CD31; B) α-EBA; also, illustrates binding specificity of SMI71 to Ngr2: C) Mock transfected (no primary); B) transfected with Ngrh1+pool (no primary); C) mock transfected (SMI71); D) transfected with Ngrh1+pool (SMI71); E) mock transfected (isotype control: GM2-50); F) transfected with Ngrh1+pool (GM2-50); EBA staining of: I) Ngr transfected; J) Ngr2 transfected; and K) Ngr3 transfected.

FIG. 25. Illustrates expression cloning of EBA antigen (similar to FIG. 18): A) and B) negative pools; C) positive pool: 2,500 clones/pool; D) positive pool: 200 clones/pool; E) positive pool: 20 clones/pool; F) positive pool: 1 clone.

FIG. 26. Illustrates splice variant of Ngrh1 expression in brain endothelial cells: A) RT-PCR on purified endothelial cells: panel 1 is spleen; panel 2 is brain; B) Predicted domain structure of Ngrh1 variants: panel 1 is Ngrh1-splice variant; panel 2 is full length Ngrh1; C) Ngrh1 western blot on fractionated rat brain: panel 1 is brain homogenate; panel 2 is brain parenchyma fraction; panel 3 is brain vessel fraction.

FIG. 27. Illustrates BBB disruption following systemic injection of MAG: A) MAG-Fc injection; B) control Fc injection.

FIG. 28. Depicts the amino acid sequences of full-length NGR2, rat NgR2 splice variant, and the nucleic acid sequence of NgRH1.

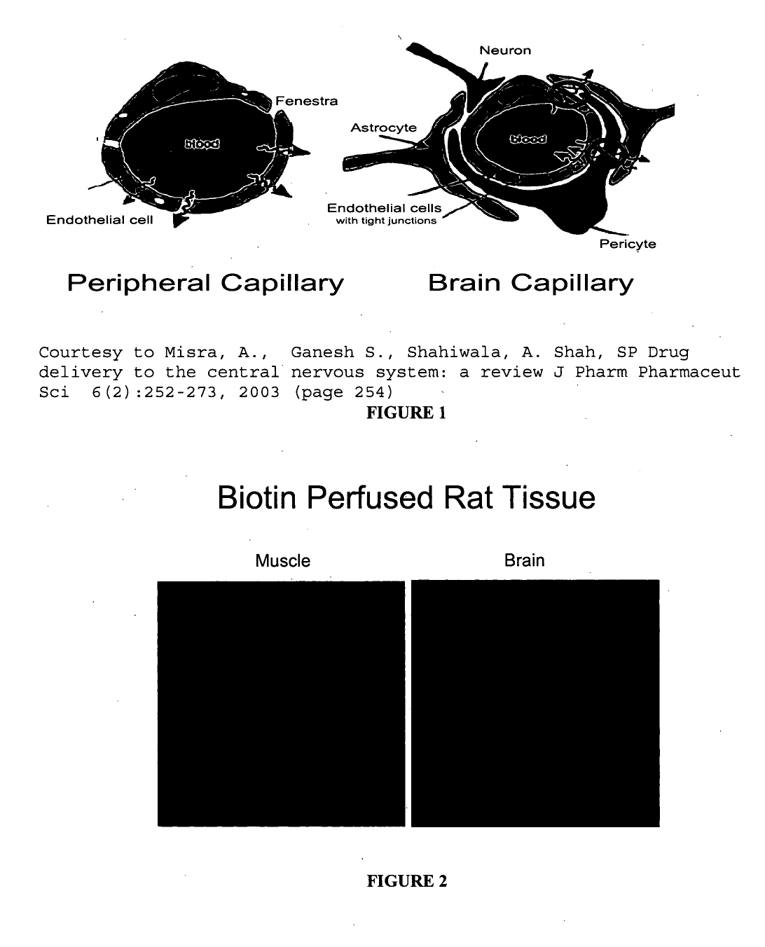

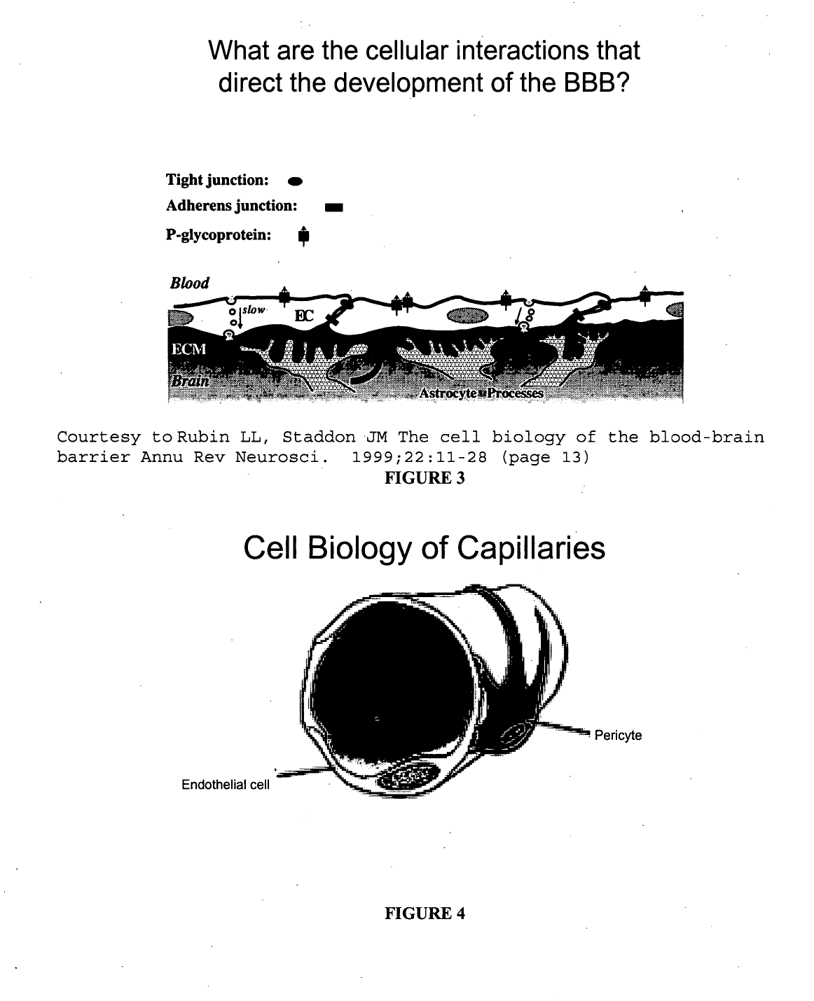

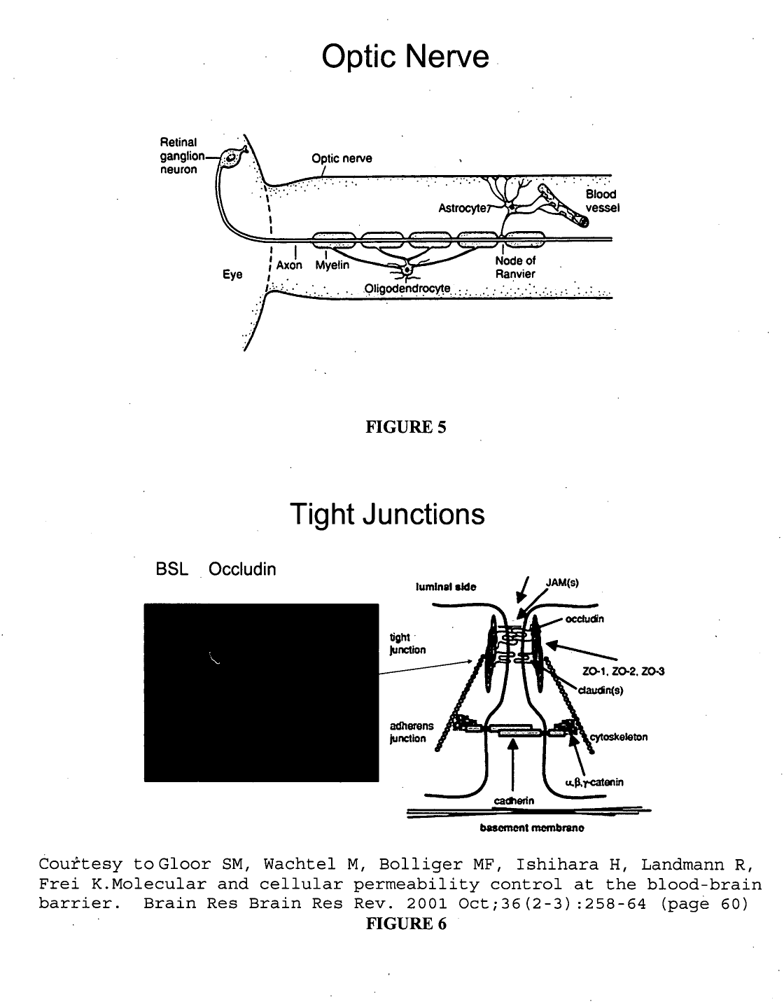

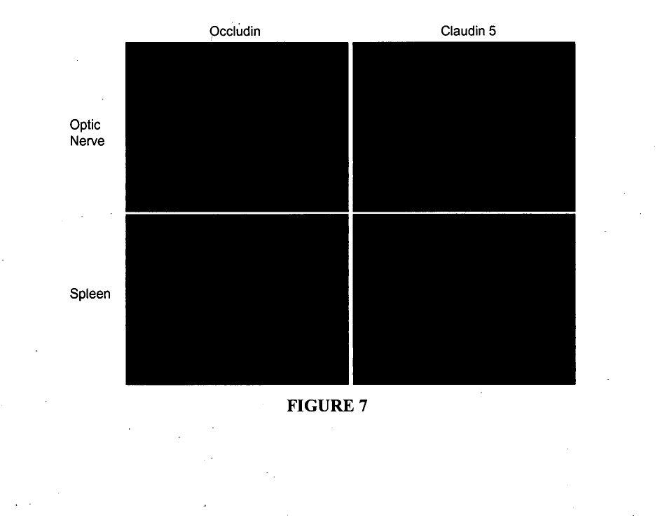

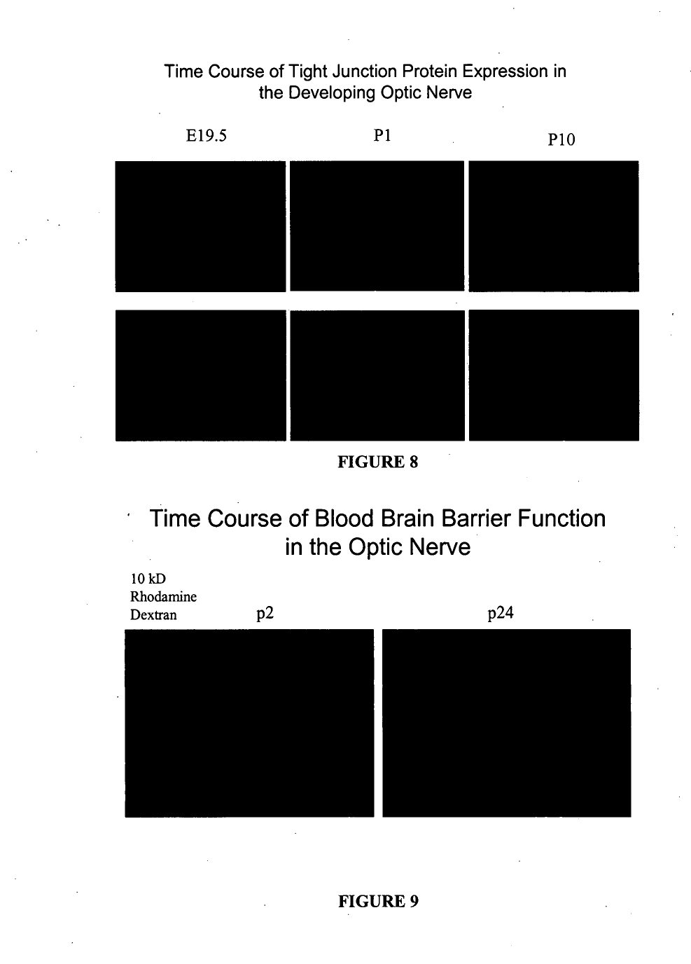

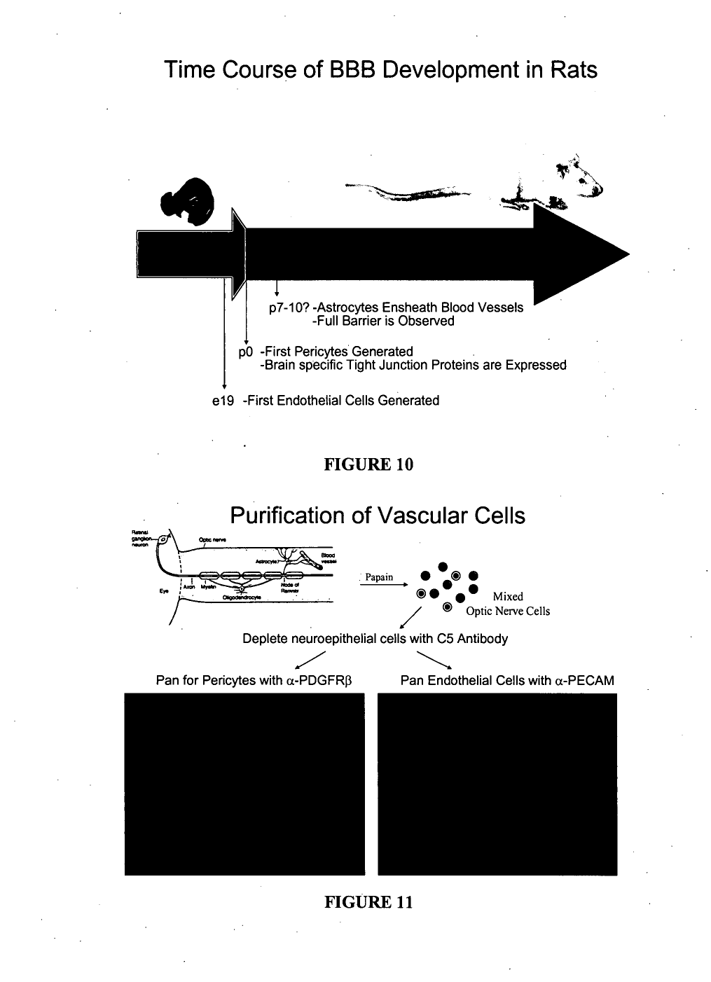

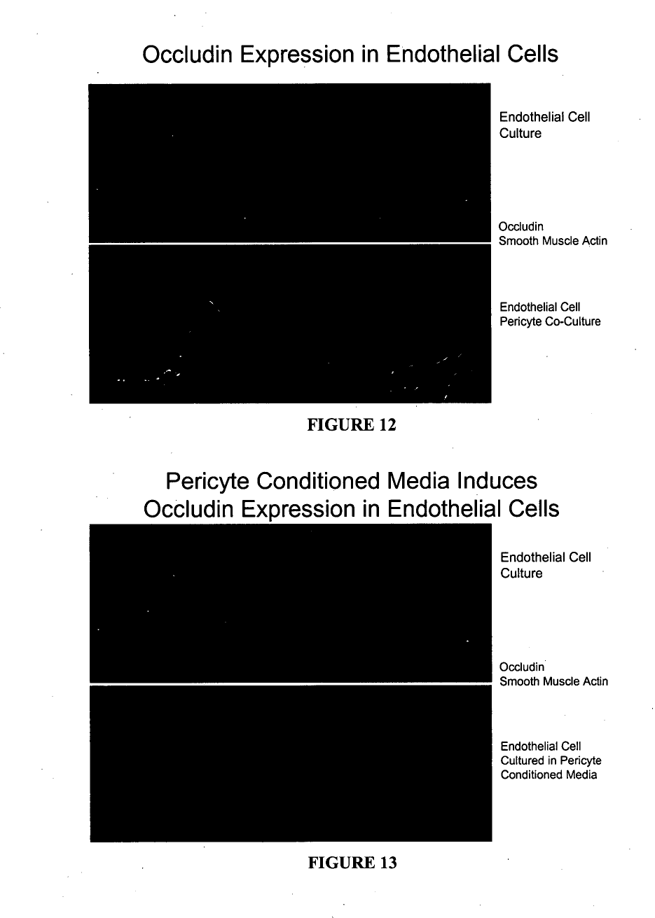

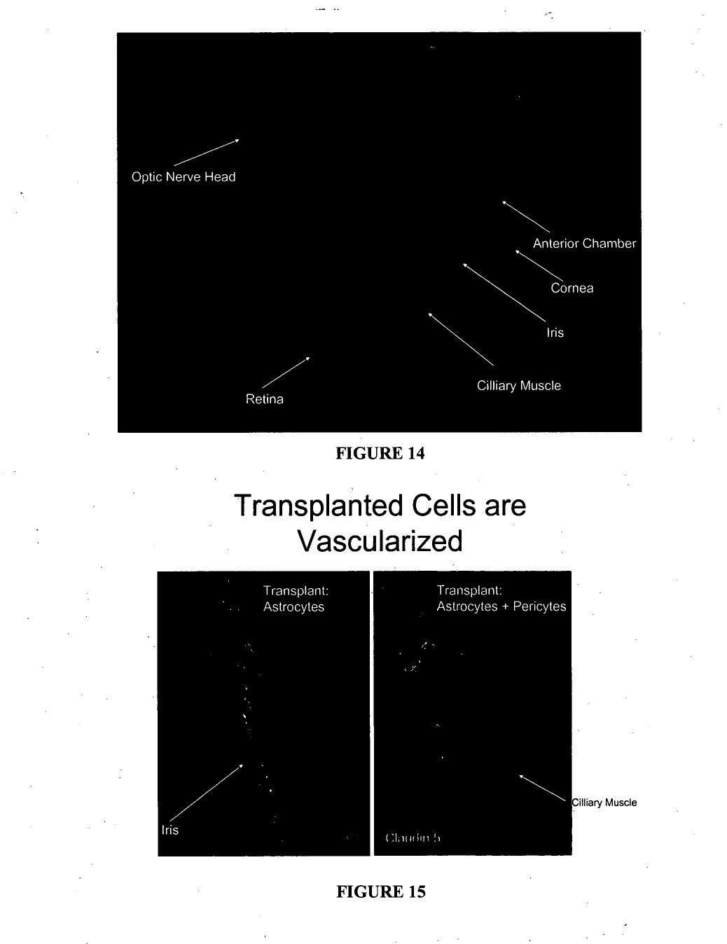

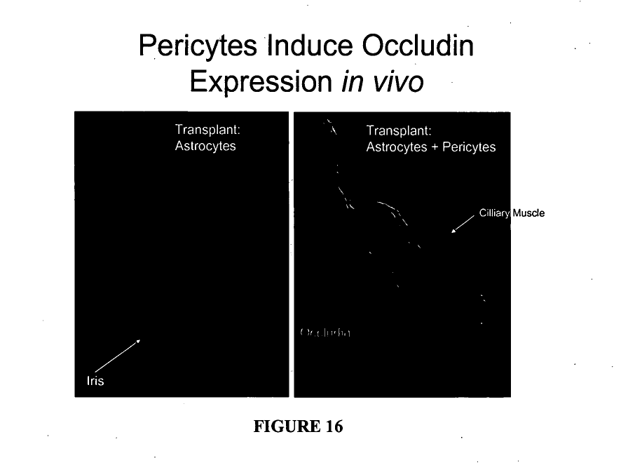

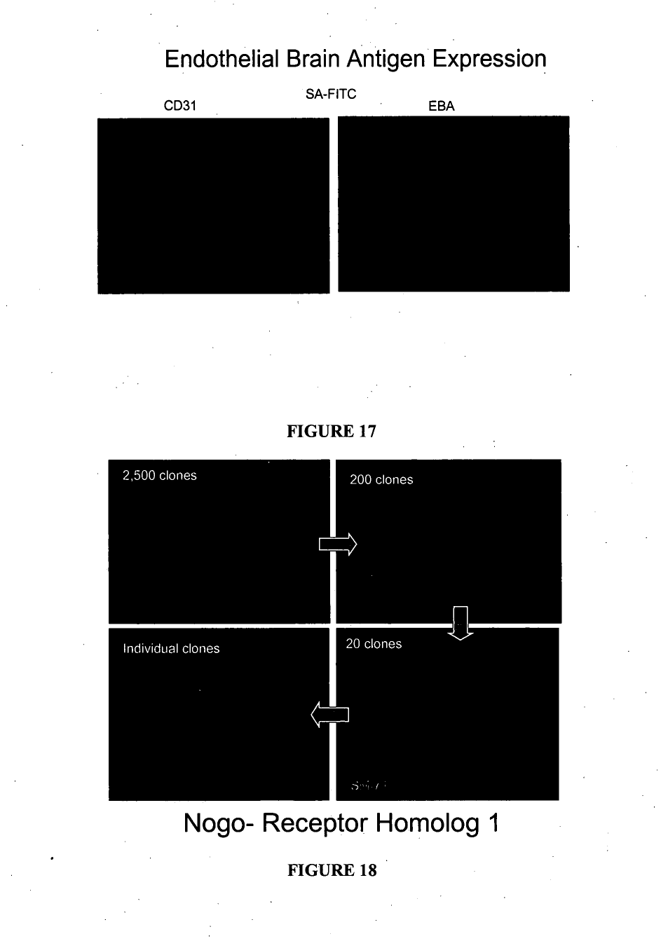

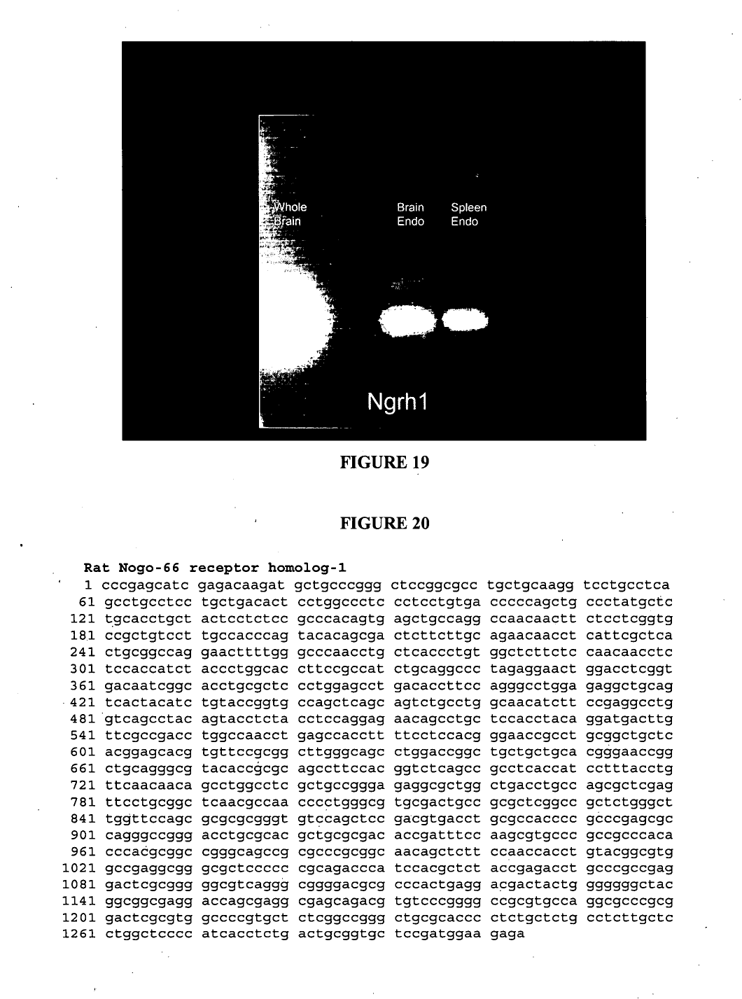

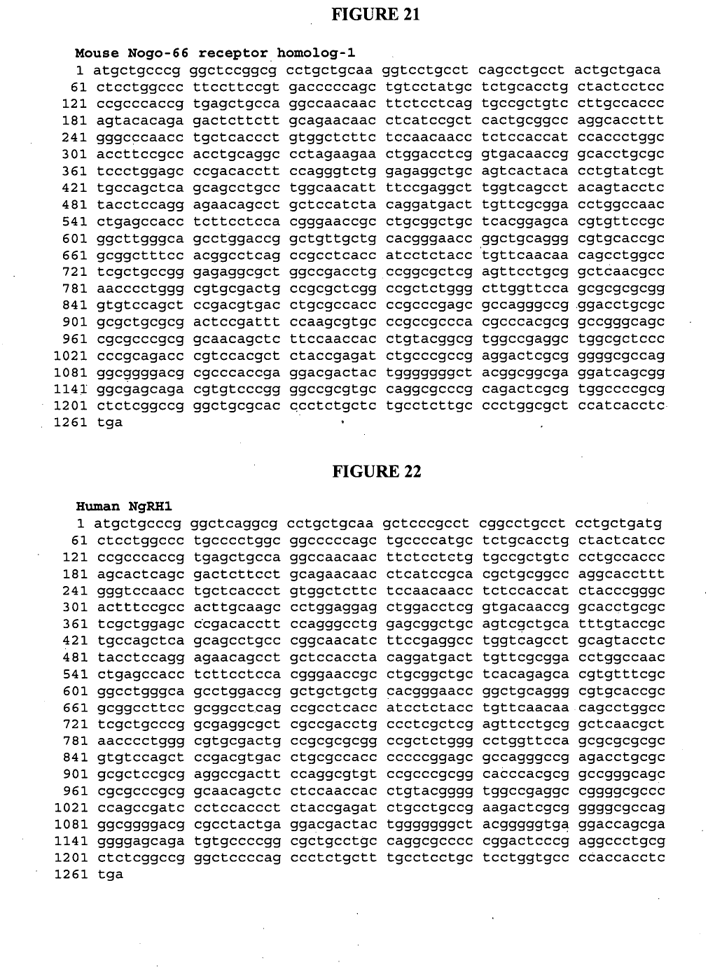

### DETAILED DESCRIPTION OF THE EMBODIMENTS

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

Other objects, features, advantages and aspects of the present invention will become apparent to those of skill from the following description. It should be understood, however, that the following description and the specific examples, while indicating preferred embodiments of the invention, are given by way of illustration only. Various changes and modifications within the spirit and scope of the disclosed invention will become readily apparent to those skilled in the art from reading the following description and from reading the other parts of the present disclosure.

General Techniques:

The practice of the present invention employs, unless otherwise indicated, conventional techniques of immunology, biochemistry, chemistry, molecular biology, microbiology, cell biology, genomics and recombinant DNA, which are within the skill of the art. See Sambrook, Fritsch and Maniatis, MOLECULAR CLONING: A LABORATORY MANUAL, 2^{nd} edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

Definitions:

As used in the specification and claims, the singular form “a”, “an” and “the” include plural references unless the context clearly dictates otherwise. For example, the term “a cell” includes a plurality of cells, including mixtures thereof.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

As used herein, “expression” refers to the process by which a polynucleotide is transcribed into mRNA and/or the process by which the transcribed mRNA (also referred to as “transcript”) is subsequently being translated into peptides, polypeptides, or proteins. The transcripts and the encoded polypeptides are collectedly referred to as “gene product.” If the polynucleotide is derived from genomic DNA, expression may include splicing of the mRNA in a eukaryotic cell.

The term “differentially expressed” as applied to nucleotide sequence or polypeptide sequence in a subject, refers to over-expression or under-expression of that sequence when compared to that detected in a control. Under-expression also encompasses absence of expression of a particular sequence as evidenced by the absence of detectable expression in a test subject when compared to a control.

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” refers to either natural and/or unnatural or synthetic amino acids, including glycine and both the D or L optical isomers, and amino acid analogs and peptidomimetics.

A “subject,” “individual” or “patient” is used interchangeably herein, which refers to a vertebrate, preferably a mammal, more preferably a human. Mammals include, but are not limited to mice (murines), rats, dogs, pigs, monkey (simians) humans, farm animals, sport animals, and pets. Tissues, cells and their progeny of a biological entity obtained in vivo or cultured in vitro are also encompassed.

As used in herein “cell” is used in its usual biological sense, and does not refer to an entire multicellular organism. The cell can, for example, be in vitro, e.g., in cell culture, or present in a multicellular organism, including, e.g., birds, plants and mammals such as humans, cows, sheep, apes, monkeys, swine, dogs, cats, mice or rats.

The terms “agent” and “biologically active agent” are used interchangeably and encompass plural references in the context stated. The “biologically active agents” that are employed in the animal model or cell culture assays described herein may be selected from the group consisting of a biological or chemical compound such as a simple or complex organic or inorganic molecule, peptide, peptide mimetic, protein (e.g. antibody), carbohydrate-containing molecule, phospholipids, liposome, small interfering RNA, or a polynucleotide (e.g. anti-sense). Furthermore, such agents include complex organic or inorganic molecules can include a heterogeneous mixture of compounds, such as crude or purified plant extracts.

The term “control” is an alternative subject, cell or sample used in an experiment for comparison purpose. Furthermore, a “control” can also represent the same subject, cell or sample in an experiment for comparison of different time points.

The term “barrier” is used in referring to the blood-brain barrier (BBB). Furthermore, the term “barrier” is also utilized to refer to cell surface proteins the are involved in BBB function.

The terms “target”, “targets”, “targeting” as applied to an agent refer to the ability of the agent to directly or indirectly affect a function or effect in the particular context used. For example, where an antibody “targets” an antigen, the antibody is specific for said antigen. In other instances, when an agent “targets” an antigen (e.g., NgRH1), it can directly or indirectly assert an effect on the antigen's activity (e.g., NgRH1 activity or signaling pathway) and/or expression via e.g., direct interaction with the antigen or indirect association with the antigen via other intermediate messenger molecules operating in the same or related pathways.

The terms “restoring”, “restored” or “restoration” are used interchangeably and mean reverting to state that is not currently present in the context of permeability.

The terms “modulating”, “modulated” or “modulation” are used interchangeably and mean a direct or indirect change in a given context. For example, modulation of permeability can be decreased or increased.

“NgRH1” (Nogo receptor homolog 1) also known “NgR2” (Nogo receptor 2) when used to refer to a protein encompass fragments of the full-length protein, alternatively spliced variants, allele homologs, that are implicated in BBB permeability.

A “BBB-related disorder” includes any disorder, condition or disease which results in the BBB as closing or opening (i.e., decreased or increased permeability) as compared to a control or reference, including a temporal control or reference in the same subject.

“Complementarity” refers to the ability of a nucleic acid to form hydrogen bond(s) with another RNA sequence by either traditional Watson-Crick or other non-traditional types. In reference to the nucleic molecules of the present invention, the binding free energy for a nucleic acid molecule with its target or complementary sequence is sufficient to allow the relevant function of the nucleic acid to proceed, e.g., enzymatic nucleic acid cleavage, antisense or triple helix inhibition. Determination of binding free energies for nucleic acid molecules is well known in the art (see, e.g., Turner et al., 1987, CSH Symp. Quant. Biol. LII pp. 123-133; Frier et al., 1986, Proc. Nat. Acad. Sci. USA 83:9373-9377; Turner et al., 1987, J. Am. Chem. Soc. 109:3783-3785). A percent complementarity indicates the percentage of contiguous residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations, amelioration of a disease, disorder or pathological condition, reducing or preventing the onset of a disease, disorder or condition, and generally counteracting a disease, disorder or pathological condition.

Modulation of BBB Permeability

In one aspect, the present invention provides a method of modulating blood-brain barrier (BBB) permeability. The method involves administering an agent to a subject, wherein the agent targets an NgRH1 cell surface receptor that is present in the brain. The method exploits, in part, the observation that NgRH1 is a barrier protein predominantly expressed in endothelial cells (in particular luminal membrane of the endothelial cells) that make up the BBB. It has been shown herein that NgRH1 is expressed in low level or even absent in leaky vessels including those in the liver, heart, lung, muscle, and the circum-ventricular organs of the brain.

In practicing the subject method, the NgRH1 target implicated in BBB permeability can be a protein having the amino acid sequence exemplified herein or any splice variant thereof (e.g., a variant encoded by the sequence exemplified in the figures attached herewith). Other NgRH1 receptors exhibiting a sequence homology of at least about 70%, 80%, 90%, 95%, 98%, or even 99% may also be targeted. One skilled in the art can ascertain the choice of target by performing a sequence alignment or conducting a homology search, which is often carried out with the aid of computer methods. In general, percent sequence identify is defined by the ratio of the number of nucleotide or amino acid matches between the query sequence and the known sequence when the two are optimally aligned. A variety of software programs are available in the art. Non-limiting examples of these programs are Blast and Fasta. Any sequence databases that contains DNA sequences corresponding to a gene or a segment thereof can be used for sequence analysis. Commonly employed databases include but are not limited to GenBank, EMBL, DDBJ, PDB, SWISS-PROT, EST, STS, GSS, and HTGS. Sequence similarity can be discerned by aligning the putative target sequence against a nucleic sequence or protein database.

The agents of choice may target NgRH1 receptor by directly binding to the receptor, or by indirectly asserting an effect on the receptor's activity or signaling pathway. In particular, the agent may indirectly associate with the receptor via other intermediate messenger molecules operating in the same or related pathways. Suitable agents that can be used to target an NgRH1 receptor include but are not limited to any biological or chemical compound such as a simple or complex organic or inorganic molecules, peptides, peptide mimetics, proteins (e.g. antibodies), carbohydrate-containing molecules, phospholipids, liposomes, small interfering RNAs, anti-sense and external guide sequences, as well as hammerhead ribozymes, DNAzymes, allozymes, aptamers, and decoys.

In yet another aspect of the invention, one or more anti-NgRH1 antibodies are administered to modulate BBB permeability in a subject. In some embodiments, one or more anti-NgRH1 antibodies are administered to a subject, utilizing the various dosage and temporal aspects for administration that are described herein.

The term “antibody” as used herein refers to immunoglobulin molecules and immunologically active portions of immunoglobulin molecules, i.e., molecules that contain an antigen-binding site which specifically binds (“immunoreacts with”) an antigen. Structurally, the simplest naturally occurring antibody (e.g., IgG) comprises four polypeptide chains, two heavy (H) chains and two light (L) chains inter-connected by disulfide bonds. The immunoglobulins represent a large family of molecules that include several types of molecules, such as IgD, IgG, IgA, IgM and IgE. The term “immunoglobulin molecule” includes, for example, hybrid antibodies, or altered antibodies, and fragments thereof. It has been shown that the antigen binding function of an antibody can be performed by fragments of a naturally-occurring antibody.

Suitable antibodies of the present invention can comprise non-single-chain antibodies and single chain antibodies. Examples of the non-single-chain antibodies include but are not limited to (i) a ccFv fragments that are described in U.S. Pat. No. 6,833,441; (ii) any other monovalent and multivalent molecules comprising at least one ccFv fragment as described herein; (iii) an Fab fragment consisting of the VL, VH, CL and CH1 domains; (iv) an Fd fragment consisting of the VH and CH1 domains; (v) an Fv fragment consisting of the VL and VH domains of a single arm of an antibody; (vi) an F(ab′)2 fragment, a bivalent fragment comprising two Fab fragments linked by a disulfide bridge at the hinge region; and (vii) a diabody.

The subject antibodies can be either “monovalent” or “multivalent.” Whereas the former has one binding site per antigen-binding unit, the latter contains multiple binding sites capable of binding to more than one antigen of the same or different kind. Depending on the number of binding sites, a subject antibody may be bivalent (having two antigen-binding sites), trivalent (having three antigen-binding sites), tetravalent (having four antigen-binding sites), and so on. Multivalent antibodies can be further classified on the basis of their binding specificities. A “monospecific” antibody is a molecule capable of binding to one or more antigens of the same kind. A “multispecific” antibody is a molecule having binding specificities for at least two different antigens.

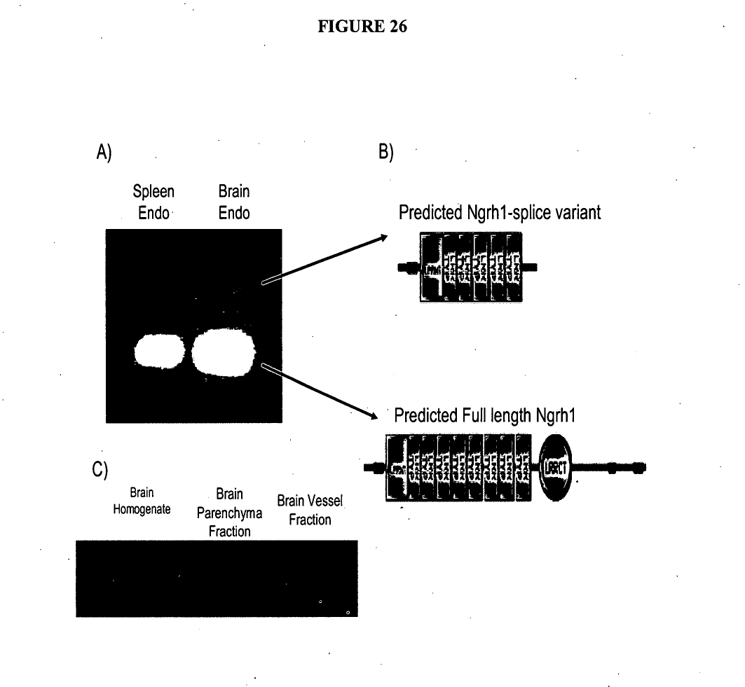

To modulate BBB permeability, one may employ a monovalent or multivalent antibody directed to any epitope of NgRH1 receptor (including a splice variant thereof). Such epitope may reside in the N-terminus, C-terminus, or in the middle of the receptor sequence, any of the leucine rich sequences (see, e.g., FIG. 26). The epitope may comprise at least 3, preferably 6, 7, 8 or more amino acids. One may also employ multispecific antibodies that carry at least one binding site for of a full-length NgRH1 receptor, and another binding site for a splice variant of NgRH1 receptor. Where desired, the multispecific antibodies may comprise a binding site for an antigen implicated in a CNS disorders, including but not limited to brain tumor and other BBB related disorders. Such configuration allows one to target an antigen implicated in BBB permeability (e.g., NgRH1 including a splice variant thereof) thereby increasing the BBB permeability and simultaneously targeting a disease antigen of interest.

Examples of bispecific antigen binding units include those with one arm directed against an antigen implicated in BBB permeability, and the other arm directed against a cytotoxic trigger molecule (to be delivered across BBB) such as anti-FcγRI/anti-CD15, anti-p185^{HER2}/IFcγRIII (CD16), anti-CD3/anti-malignant B-cell (1D10), anti-CD3/anti-p185^{HER2}, anti-CD3/anti-p97, anti-CD3/anti-renal cell carcinoma, anti-CD3/anti-OVCAR-3, anti-CD3/L-D1 (anti-colon carcinoma), anti-CD3/anti-melanocyte stimulating hormone analog, anti-EGF receptor/anti-CD3, anti-CD3/anti-CAMA1, anti-CD3/anti-CD19, anti-CD3/MoV18, anti-neural cell ahesion molecule (NCAM)/anti-CD3, anti-folate binding protein (FBP)/anti-CD3, anti-pan carcinoma associated antigen (AMOC-31)/anti-CD3; bispecific antibodies with one arm which binds specifically to a tumor antigen and one arm which binds to a toxin such as anti-saporin/anti-Id-1, anti-CD22/anti-saporin, anti-CD7/anti-saporin, anti-CD38/anti-saporin, anti-CEA/anti-ricin A chain, anti-interferon-α (IFN-α)/anti-hybridoma idiotype, anti-CEA/anti-vinca alkaloid; BsAbs for converting enzyme activated prodrugs such as anti-CD30/anti-alkaline phosphatase (which catalyzes conversion of mitomycin phosphate prodrug to mitomycin alcohol); bispecific antibodies which can be used as fibrinolytic agents such as anti-fibrin/anti-tissue plasminogen activator (tPA), anti-fibrin/anti-urokinase-type plasminogen activator (uPA); bispecific antigen-binding untis for targeting immune complexes to cell surface receptors such as anti-low density lipoprotein (LDL)/anti-Fc receptor (e.g. Fcγ RI, FcγRII or FcγRIII); bispecific antibodies for use in therapy of infectious diseases such as anti-CD3/anti-herpes simplex virus (HSV), anti-T-cell receptor:CD3 complex/anti-influenza, anti-FcγR/anti-HIV; bispecific antibodies for tumor detection in vitro or in vivo such as anti-CEA/anti-EOTUBE, anti-CEA/anti-DPTA, anti-p185^{HER2}/anti-hapten; BsAbs as vaccine adjuvants (see Fanger et al., supra); and bispecific antibodies as diagnostic tools such as anti-rabbit IgG/anti-ferritin, anti-horse radish peroxidase (HRP)/anti-hormone, anti-somatostatin/anti-substance P, anti-HRP/anti-FITC, anti-CEA/anti-.beta.-galactosidase (see Nolan et al., supra). Examples of trispecific antibodies include anti-CD3/anti-CD4/anti-CD37, anti-CD3/anti-CD5/anti-CD37 and anti-CD3/anti-CD8/anti-CD37.

Suitable antibodies are immunoglobulin molecules of a variety of species origins including invertebrates and vertebrates. Preferred antibodies include human antibodies that are expressed by a human gene(s) or fragment(s) thereof. Antibodies can also be humanized as applies to a non-human (e.g. rodent or primate) antibodies are hybrid immunoglobulins, immunoglobulin chains or fragments thereof which contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, rabbit or primate having the desired specificity, affinity and capacity. In some instances, Fv framework region (FR) residues of the human immunoglobulin are replaced by corresponding non-human residues. Furthermore, the humanized antibody may comprise residues which are found neither in the recipient antibody nor in the imported CDR or framework sequences. These modifications are made to further refine and optimize antibody performance and minimize immunogenicity when introduced into a human body. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody may also comprise at least a portion of an immunoglobulin constant region (Fc), typically that of a human immunoglobulin.

Methods for humanizing non-human antibodies are well known in the art. “Humanized” antibodies are antibodies in which at least part of the sequence has been altered from its initial form to render it more like human immunoglobulins. In one version, the H chain and L chain C regions are replaced with human sequence. This is a fusion polypeptide comprising a V region and a heterologous immunoglobulin C region. In another version, the CDR regions comprise non human antibody sequences, while the V framework regions have also been converted human sequences. See, for example, EP 0329400. In a third version, V regions are humanized by designing consensus sequences of human and mouse V regions, and converting residues outside the CDRs that are different between the consensus sequences.

In making humanized antibodies, the choice of framework residues can be important in retaining high binding affinity. In principle, a framework sequence from any HuAb can serve as the template for CDR grafting; however, it has been demonstrated that straight CDR replacement into such a framework can lead to significant loss of binding affinity to the antigen. Glaser et al. (1992) J. Immunol. 149:2606; Tempest et al. (1992) Biotechnology 9:266; and Shalaby et al. (1992) J. Exp. Med. 17:217. The more homologous a HuAb is to the original muAb, the less likely that the human framework will introduce distortions into the murine CDRs that could reduce affinity. Based on a sequence homology search against an antibody sequence database, the HuAb IC4 provides good framework homology to muM4TS.22, although other highly homologous HuAbs would be suitable as well, especially kappa L chains from human subgroup I or H chains from human subgroup III. Kabat et al. (1987). Various computer programs such as ENCAD (Levitt et al. (1983) J. Mol. Biol. 168:595) are available to predict the ideal sequence for the V region. The invention thus encompasses HuAbs with different V regions. It is within the skill of one in the art to determine suitable V region sequences and to optimize these sequences. Methods for obtaining antibodies with reduced immunogenicity are also described in U.S. Pat. No. 5,270,202 and EP 699,755.

Where desired, the antibodies are humanized with retention of high affinity for the antigen and other favorable biological properties. To achieve this goal, according to a preferred method, humanized antibodies are prepared by a process of analysis of the parental sequences and various conceptual humanized products using three dimensional models of the parental and humanized sequences. Three dimensional immunoglobulin models are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved.

The invention also encompasses antibodies conjugated to a chemically functional moiety. Typically, the moiety is a label capable of producing a detectable signal. These conjugated antibodies are useful, for example, in detection systems such as quantitation of myelin lesions, tumor burden, and imaging of metastatic foci and tumor imaging. Such labels are known in the art and include, but are not limited to, radioisotopes, enzymes, fluorescent compounds, chemiluminescent compounds, bioluminescent compounds substrate cofactors and inhibitors. See, for examples of patents teaching the use of such labels, U.S. Pat. Nos. 3,817,837; 3,850,752; 3,939,350; 3,996,345; 4,277,437; 4,275,149; and 4,366,241. The moieties can be covalently linked to antibodies, recombinantly linked, or conjugated to antibodies through a secondary reagent, such as a second antibody, protein A, or a biotin-avidin complex.

Other functional moieties include signal peptides, agents that enhance immunologic reactivity, agents that facilitate coupling to a solid support, vaccine carriers, bioresponse modifiers, paramagnetic labels and drugs. Signal peptides is a short amino acid sequence that directs a newly synthesized protein through a cellular membrane, usually the endoplasmic reticulum in eukaryotic cells, and either the inner membrane or both inner and outer membranes of bacteria. Signal peptides are typically at the N-terminal portion of a polypeptide and are typically removed enzymatically between biosynthesis and secretion of the polypeptide from the cell. Such a peptide can be incorporated into the subject antibodies to allow secretion of the synthesized molecules

An additional aspect of the invention is directed to administering a soluble antigen that is also specific for one or more anti-NgRH1 antibodies, whereby said one or more anti-NgRH1 antibodies can bind both said antigen and said NgRH1 cell surface receptor. In a preferred embodiment, said antigen has or is modified to have, either greater or less binding specificity for said one or more anti-NgRH1 antibodies, as compared to the targeted NgRH1 cell surface receptor and the one or more anti-NgRH1 antibodies. In some embodiments, said antigen is administered before, concomitantly or after said one or more antibodies are administered to a subject.

In other aspects, said soluble antigen is designed to also bind NgRH1 cell surface receptor with greater or less binding specificity as compared to said one or more anti-NgRH1 antibodies bind said NgRH1 cell surface receptor. In one embodiment, the antigen competes with one or more anti-NgRH1 antibodies to bind said NgRH1 cell surface receptor. In another embodiment, said soluble antigen is administered before, concomitantly or after said one or more anti-NgRH1 antibodies are administered to a subject.

Such soluble antigens with altered NgRH1 or anti-NgRH1 antibodies can be selected or engineered utilizing proteomic computational analysis methods available in the art. Essentially such analysis is utilized to select or engineer proteins (i.e., soluble antigens) that have different binding kinetics as compared to naturally occurring binding pairs. See, Kortemme and Baker. Curr. Opin. Chem. Biol. 2004; 8:91-97; U.S. Pat. No. 6,951,715.

Other bioactive peptides (or antigens) useful according to the invention may be identified through the use of synthetic peptide combinatorial libraries such as those disclosed in Houghton et al., Biotechniques, 13(3):412-421 (1992) and Houghton et al., Nature, 354:84-86 (1991) or using phage display procedures such as those described in Hart, et al., J. Biol. Chem. 269:12468 (1994). Hart et al. report a filamentous phage display library for identifying novel peptide ligands for mammalian cell receptors. In general, phage display libraries using, e.g., M13 or fd phage, are prepared using conventional procedures such as those described in the foregoing reference. The libraries display inserts containing from 4 to 80 amino acid residues. The inserts optionally represent a completely degenerate or a biased array of peptides. Ligands that bind selectively to a specific molecule such as a cell surface receptor are obtained by selecting those phages which express on their surface a ligand that binds to the specific molecule. Ligands that possess a desired biological activity can be screened in known biological activity assays and selected on that basis. These phages then are subjected to several cycles of reselection to identify the peptide-expressing phages that have the most useful characteristics. Typically, phages that exhibit the binding characteristics (e.g., highest binding affinity or cell stimulatory activity) are further characterized by nucleic acid analysis to identify the particular amino acid sequences of the peptides expressed on the phage surface and the optimum length of the expressed peptide to achieve optimum biological activity.

Alternatively, such peptides can be selected from combinatorial libraries of peptides containing one or more amino acids. Such libraries can further be synthesized which contain non-peptide synthetic moieties which are less subject to enzymatic degradation compared to their naturally-occurring counterparts. U.S. Pat. No. 5,591,646 discloses methods and apparatuses for biomolecular libraries which are useful for screening and identifying bioactive peptides. Methods for screening peptides libraries are also disclosed in U.S. Pat. No. 5,565,325. Peptides obtained from combinatorial libraries or other sources can be screened for functional activity by methods known in the art. Therefore, one of skill can identify peptides that have some level of specificity for NgRH1 and such peptides can be utilized to modulate BBB premeability.

Aptamers targeting NgRH1 represent a particularly useful class of agents. Aptamers include DNA, RNA or peptides that are selected based on specific binding properties to a particular molecule. For example, an aptamer(s) can be selected for binding NgRH1 using methods known in the art. Subsequently, said aptamer(s) can be administered to a subject to modulate BBB permeability. Some aptamers having affinity to a specific protein, DNA, amino acid and nucleotides have been described (e.g., K. Y. Wang, et al., “A DNA Aptamer Which Binds to and Inhibits Thrombin Exhibits a New Structural Motif for DNA,” Biochemistry 32:1899-1904 (1993); Pitner et al., U.S. Pat. No. 5,691,145; Gold, et al., “Diversity of Oligonucleotide Function,” Ann. Rev. Biochem. 64: 763-97 (1995); Szostak et al., U.S. Pat. No. 5,631,146). High affinity and high specificity binding aptamers have been derived from combinatorial libraries (supra, Gold, et al.). Aptamers may have high affinities, with equilibrium dissociation constants ranging from micromolar to sub-nanomolar depending on the selection used. Aptamers may also exhibit high selectivity, for example, showing a thousand fold discrimination between 7-methylG and G (Haller, A. A., and Samow, P., “In Vitro Selection of a 7-Methyl-Guanosine Binding RNA That Inhibits Translation of Capped mRNA molecules, PNAS USA 94:8521-8526 (1997)) or between D and L-tryptophan (supra, Gold et al).

General methods for screening randomized oligonucleotides for aptamer activity have been described. For example, Gold, et al. (U.S. Pat. No. 5,270,163) describes the “SELEX” (Systematic Evolution of Ligands by Exponential Enrichment) method. In Gold et al., a candidate mixture of single stranded nucleic acid having regions of randomized sequence is contacted with a target molecule. Those nucleic acids having an increased affinity to the target are partitioned from the remainder of the candidate mixture. The partitioned nucleic acids are amplified to yield a ligand enriched mixture. Szostak et al. (U.S. Pat. No. 5,631,146) describes a method for producing a single stranded DNA molecule which binds adenosine or an adenosine-5′-phosphate.

Aptamers according to this invention may be modified to improve binding specificity or stability as long as the aptamer retains a portion of its ability to bind and recognize its target monomer. For example, methods for modifying the bases and sugars of nucleotides are known in the art. Typically, phosphodiester linkages exist between the nucleotides of an RNA or DNA. An aptamer according to this invention may have phosphodiester, phosphoroamidite, phosphorothioate or other known linkages between its nucleotides to increase its stability provided that the linkage does not substantially interfere with the interaction of the aptamer with its target monomer.

An aptamer suitable for use in the methods of this invention may be synthesized by a polymerase chain reaction (PCR), a DNA or RNA polymerase, a chemical reaction or a machine according to standard methods known in the art. For example, an aptamer may be synthesized by an automated DNA synthesizer from Applied Biosystems, Inc. (Foster City, Calif.) using standard chemistries.

In addition, aptamer binding to NgRH1 can be optimized post-selection. For example, one modification is “stickiness” of thio- and dithio-phosphate ODN agents to enhance the affinity and specificity to a protein target. In a significant improvement over existing technology, the method of selection concurrently controls and optimizes the total number of thiolated phosphates to decrease non-specific binding to non-target proteins and to enhance only the specific favorable interactions with the target. Therefore selected aptamers used in methods of the present invention can be modified to permit the selective development of aptamers that have the combined attributes of affinity, specificity and nuclease resistance. Such optimization methods are known in the art, such as in the disclosure of U.S. Pat. No. 6,867,289.

The agent can also take the form of a decoy. By “decoy” is meant a nucleic acid molecule, for example RNA or DNA, or aptamer that is designed to preferentially bind to a predetermined ligand or unknown ligand. Such binding can result in the inhibition or activation of a target molecule. The decoy or aptamer can compete with a naturally occurring binding target for the binding of a specific ligand. For example, it has been shown that over-expression of HIV trans-activation response (TAR) RNA can act as a “decoy” and efficiently binds HIV tat protein, thereby preventing it from binding to TAR sequences encoded in the HIV RNA (Sullenger et al., 1990, Cell, 63, 601-608). This is but a specific example and those in the art will recognize that other embodiments can be readily generated using techniques generally known in the art, see for example Gold et al., 1995, Annu. Rev. Biochem., 64, 763; Brody and Gold, 2000, J. Biotechnol., 74, 5; Sun, 2000, Curr. Opin. Mol. Ther., 2, 100; Kusser, 2000, J. Biotechnol., 74, 27; Hermann and Patel, 2000, Science, 287, 820; and Jayasena, 1999, Clinical Chemistry, 45, 1628. Similarly, a decoys can be designed to bind to NgRH1 or NgRH2 and block the binding of the same, or a decoy can be designed to bind to NgRH1 and prevent interaction with another ligand protein(s).

Where desired, the agent of the present invention is in the form of siRNA. An siRNA molecule of the invention typically comprises a double stranded RNA wherein one strand of the RNA is complimentary to the RNA of the NgRH1 or NgRH2 gene. In another embodiment, a siRNA molecule of the invention comprises a double stranded RNA wherein one strand of the RNA comprises a portion of a sequence of RNA having NgRH1 or NgRH2 gene sequence. In yet another embodiment, a siRNA molecule of the invention comprises a double stranded RNA wherein both strands of RNA are connected by a non-nucleotide linker. Alternatively, an siRNA molecule of the invention comprises a double stranded RNA wherein both strands of RNA are connected by a nucleotide linker, such as a loop or stem loop structure.

Standard methods in the design of siRNA are known in the art (Elbashir et al., Methods 26:199-213 (2002)). In general, a suitable siRNA is between about 10-50, or about 20-25 nucleotides, or about 20-22 nucleotides. The target site typically has an AA dinucleotide at the 3′ end of the sequence, as siRNA with a UU overhang can be more effective in gene silencing. The remaining nucleotides generally exhibit homology to the nucleotides 3′ of the AA dinucleotides. In general, the siRNA typically exhibits at least about 50% homology to the target sequence, e.g., NgRH1 receptor sequence, preferably at least about 70%, about 80%, 90% or even 95% homology to the target sequence. Where desired, potential target sites are also compared to the appropriate genome database, such that target sequences should have fewer than 70%, 60%, 50%, 40%, 30%, 20%, 10%, 5%, or even 1% homology to other genes. The readily available public database on the NCBI server, www.ncbi.nlm.nih.gov/BLAST is a convenient tool used to determine sequence homology. A public siRNA design tool is also readily available from the Whitehead Institute of Biomedical Research at MIT, http://jura.wi.mit.edu/pubint/http://iona.wi.mit.edu/siRNAext/.

A suitable siRNA can also take the form of a hairpin siRNA. One may vary a number of known factors in designing a suitable hairpin siRNA. Such variables include the length of the inverted repeats that encode the stem of a putative hairpin, the order of the inverted repeats, the length and composition of the spacer sequence that encodes the loop of the hairpin, and the presence or absence of 5′-overhang can vary depending on the target and the predicted inverted region; all of which can be varied or customized according to standard procedures in the art. The stem can be 19 to 20 nucleotides long, preferably about 19, 21, 25, or 29 nucleotides long. The loop can range from 3 nucleotides to 23 nucleotides, with preference for loop sizes of about 3, 4, 5, 6, 7 and 9 nucleotides. Databases available to the public to aid in the selection and design of hairpin siRNA are also available, such as www.RNAinterference.org, and online design tools, for both hairpin siRNA and siRNA are available from commerical sites such as Promega and Ambion.

Another class of agents useful for practising the subject method is external guide sequence (EGS). EGS can be used as a gene silencing agent, applicable for downregulating the function of NgRH1 or upregulating the function of NgRH1 by inhibiting the negative regulators of NgRH1. An EGS is designed to base pair through hydrogen bonding mechanism with a target mRNA to form a molecular structure similar to that of a transfer RNA (tRNA). The EGS/mRNA target is then cleaved and inactivated by RNase P. RNase P is present in abundant quantities in all viable eukaryotic cells where it serves to process transfer RNA (tRNA) by cleavage of a precursor transcript. In general, EGS is designed to mimic certain structural features of a tRNA molecule when it forms a bimolecular complex with another RNA sequence contained within a cellular messenger RNA (mRNA). Thus, any mRNA can in principle be recognized as a substrate for RNase P with both the EGS and RNase P participating as cocatalysts. Preferred EGS for eukaryotic RNAase P consist of a sequence which, when in a complex with the target RNA molecule, forms a secondary structure resembling that of a tRNA cloverleaf or parts thereof. The desired secondary structure is determined using conventional Watson-Crick base pairing schemes to form a structure resembling a tRNA. Since RNase P recognizes structures as opposed to sequences, the specific sequence of the hydrogen bonded regions is less critical than the desired structure to be formed. The EGS and the target RNA substrate should resemble a sufficient portion of the tRNA secondary and tertiary structure to result in cleavage of the target RNA by RNAase P. The sequence of the EGS can be derived from any tRNA.

Of particular interest is EGS comprising the mRNA primary sequence of NgRH1 (including a splice variant thereof). Various methods of designing EGS are described in, e.g., U.S. Pat. No. 5,624,824 to Yuan, et al., U.S. Pat. No. 6,610,478 to Takle, et al., WO 93/22434, WO 95/24489, and WO 96/21731.

Yet another class of agents are antisense molecules targeting NgRH1. Exemplary antisense nucleic acids or antisense molecules are non-enzymatic nucleic acid molecules that bind to target RNA by means of RNA-RNA or RNA-DNA or RNA-PNA (protein nucleic acid; Egholm et al., 1993 Nature 365, 566) interactions and alter the activity of the target RNA (for a review, see Stein and Cheng, 1993 Science 261, 1004 and Woolf et al., U.S. Pat. No. 5,849,902). Typically, antisense molecules are complementary to a target sequence along a single contiguous sequence of the antisense molecule. However, in certain embodiments, an antisense molecule can bind to substrate such that the substrate molecule forms a loop, and/or an antisense molecule can bind such that the antisense molecule forms a loop. Thus, the antisense molecule can be complementary to two (or even more) non-contiguous substrate sequences or two (or even more) non-contiguous sequence portions of an antisense molecule can be complementary to a target sequence or both. For a review of current antisense strategies, see Schmajuk et al., 1999, J. Biol. Chem., 274, 21783-21789, Delihas et al., 1997, Nature, 15, 751-753, Stein et al., 1997, Antisense N. A. Drug Dev., 7, 151, Crooke, 2000, Methods Enzymol., 313, 3-45; Crooke, 1998, Biotech. Genet. Eng. Rev., 15, 121-157, Crooke, 1997, Ad. Pharmacol, 40, 1-49. In addition, antisense DNA can be used to target RNA by means of DNA-RNA interactions, thereby activating RNase H, which digests the target RNA in the duplex. The antisense oligonucleotides can comprise one or more RNAse H activating region, which is capable of activating RNAse H cleavage of a target RNA. Antisense DNA can be synthesized chemically or expressed via the use of a single stranded DNA expression vector or equivalent thereof. This is a non-limiting example and those in the art will recognize that other embodiments can be readily generated using techniques generally known in the art, see for example Gold et al., U.S. Pat. Nos. 5,475,096 and 5,270,163; Gold et al., 1995, Annu. Rev. Biochem., 64, 763; Brody and Gold, 2000, J. Biotechnol., 74, 5; Sun, 2000, Curr. Opin. Mol. Ther., 2, 100; Kusser, 2000, J. Biotechnol., 74, 27; Hermann and Patel, 2000, Science, 287, 820; and Jayasena, 1999, Clinical Chemistry, 45,

Where desired, RNase H activating region can be engineered into a nucleic acid agent. RNase H activating region is a region (generally greater than or equal to 4-25 nucleotides in length, preferably from 5-11 nucleotides in length) of a nucleic acid molecule capable of binding to a target RNA to form a non-covalent complex that is recognized by cellular RNase H enzyme (see for example Arrow et al., U.S. Pat. No. 5,849,902; Arrow et al., U.S. Pat. No. 5,989,912). The RNase H enzyme binds to the nucleic acid molecule-target RNA complex and cleaves the target RNA sequence. The RNase H activating region comprises, for example, phosphodiester, phosphorothioate (preferably at least four of the nucleotides are phosphorothiote substitutions; more specifically, 4-11 of the nucleotides are phosphorothiote substitutions); phosphorodithioate, 5′-thiophosphate, or methylphosphonate backbone chemistry or a combination thereof. In addition to one or more backbone chemistries described above, the RNase H activating region can also comprise a variety of sugar chemistries. For example, the RNase H activating region can comprise deoxyribose, arabino, fluoroarabino or a combination thereof, nucleotide sugar chemistry. Those skilled in the art will recognize that the foregoing are non-limiting examples and that any combination of phosphate, sugar and base chemistry of a nucleic acid that supports the activity of RNase H enzyme is within the scope of the definition of the RNase H activating region and the instant invention

Where desired, an enzymatic nucleic acid molecule, antisense nucleic acid molecule, decoy RNA, dsRNA, siRNA, EGS, or aptamer molecule targeting NgRH1, comprises at least one 2′-sugar modification. In another embodiment, said molecules comprise at least one nucleic acid modification. In another embodiment, said molecules comprise at least one backbone modification.

In another embodiment, an enzymatic nucleic acid or antisense nucleic acid molecule or other nucleic acid molecule of the invention targeting NgRH1, comprises a cap structure, wherein the cap structure is at the 5′-end, or 3′-end, or both the 5′-end and the 3′-end, for example a 3′,3′-linked or 5′,5′-linked deoxyabasic derivative.

In yet another embodiment of the present invention, a nucleic acid molecule of the instant invention can be between about 10 and 100 nucleotides in length. For example, enzymatic nucleic acid molecules of the invention are preferably between about 15 and 50 nucleotides in length, more preferably between about 25 and 40 nucleotides in length, e.g., 34, 36, or 38 nucleotides in length (for example see Jarvis et al., 1996, J. Biol. Chem., 271, 29107-29112). Exemplary DNAzymes of the invention are preferably between about 15 and 40 nucleotides in length, more preferably between about 25 and 35 nucleotides in length, e.g., 29, 30, 31, or 32 nucleotides in length (see for example Santoro et al., 1998, Biochemistry, 37, 13330-13342; Chartrand et al., 1995, Nucleic Acids Research, 23, 4092-4096). Exemplary antisense molecules of the invention are preferably between about 15 and 75 nucleotides in length, more preferably between about 20 and 35 nucleotides in length, e.g., 25, 26, 27, or 28 nucleotides in length (see for example Woolf et al., 1992, PNAS., 89, 7305-7309; Milner et al., 1997, Nature Biotechnology, 15, 537-541). Exemplary triplex forming oligonucleotide molecules of the invention are preferably between about 10 and 40 nucleotides in length, more preferably between about 12 and 25 nucleotides in length, e.g., 18, 19, 20, or 21 nucleotides in length (see for example Maher et al, 1990, Biochemistry, 29, 8820-8826; Strobel and Dervan, 1990, Science, 249, 73-75). Those skilled in the art will recognize that all that is required is that the nucleic acid molecule be of sufficient length and suitable conformation for the nucleic acid molecule to interact with its target and/or catalyze a reaction contemplated herein, i.e., NgRH1-encoding DNA or RNA. The length of the nucleic acid molecules of the instant invention are not limiting within the general limits stated.

Preferably, a nucleic acid molecule that modulates, for example, down-regulates NgRH1 expression comprises between 12 and 100 bases complementary to a RNA molecule of NgRH1. Even more preferably, a nucleic acid molecule that modulates, for example NgRH1 expression comprises between 14 and 24 bases complementary to a RNA molecule of NgRH1.

The nucleic acid agents of the present invention can be delivered to a cell via an expression vector. Accordingly, the subject vector comprises a nucleic acid sequence of at least one enzymatic nucleic acid molecule, antisense, or other nucleic acid molecule of the invention in a manner which allows replication and/or expression of the nucleic acid molecule in cells, such as endothelial cells. Where desired, an expression vector of the invention comprises a nucleic acid sequence encoding two or more enzymatic nucleic acid molecules, which can be the same or different.

Methods of designing expression vector are known in the art. Where desired, certain of the nucleic acid molecules of the instant invention can be expressed within cells from eukaryotic promoters (e.g., Izant and Weintraub, 1985, Science, 229, 345; McGarry and Lindquist, 1986, Proc. Natl. Acad. Sci., USA 83, 399; Scanlon et al., 1991, Proc. Natl. Acad. Sci. USA, 88, 10591-5; Kashani-Sabet et al., 1992, Antisense Res. Dev., 2, 3-15; propulic et al., 1992, J. Virol., 66, 143241; Weerasinghe et al., 1991, J. Virol., 65, 5531-4; Ojwang et al., 1992, Proc. Natl. Acad. Sci. USA, 89, 10802-6; Chen et al., 1992, Nucleic Acids Res., 20, 4581-9; Sarver et al., 1990 Science, 247, 1222-1225; Thompson et al, 1995, Nucleic Acids Res., 23, 2259; Good et al., 1997, Gene Therapy, 4, 45; all of these references are hereby incorporated in their totalities by reference herein). Those skilled in the art realize that any nucleic acid can be expressed in eukaryotic cells from the appropriate DNA/RNA vector. The activity of such nucleic acids can be augmented by their release from the primary transcript by a enzymatic nucleic acid (Draper et al., PCT WO 93/23569, and Sullivan et al., PCT WO 94/02595; Ohkawa et al., 1992, Nucleic Acids Symp. Ser., 27, 15-6; Taira et al., 1991, Nucleic Acids Res., 19, 5125-30; Ventura et al., 1993, Nucleic Acids Res., 21, 3249-55; Chowrira et al., 1994, J. Biol. Chem., 269, 25856; all of these references are hereby incorporated in their totalities by reference herein). Gene therapy approaches specific to the CNS are described by Blesch et al., 2000, Drug News Perspect., 13, 269-280; Peterson et al., 2000, Cent. Nerv. Syst. Dis., 485-508; Peel and Klein, 2000, J. Neurosci. Methods, 98, 95-104; Hagihara et al., 2000, Gene Ther., 7, 759-763; and Herrlinger et al., 2000, Methods Mol. Med., 35, 287-312. AAV-mediated delivery of nucleic acid to cells of the nervous system is further described by Kaplitt et al., U.S. Pat. No. 6,180,613.

Vectors utilized in in vivo or in vitro methods can include derivatives of SV-40, adenovirus, retrovirus-derived DNA sequences and shuttle vectors derived from combinations of functional mammalian vectors and functional plasmids and phage DNA. Eukaryotic expression vectors are well known, e.g. such as those described by P J Southern and P Berg, J Mol Appl Genet. 1:327-341 (1982); Subramini et al., Mol. Cell. Biol. 1:854-864 (1981), Kaufmann and Sharp, J. Mol. Biol. 159:601-621 (1982); Scahill et al., PNAS USA 80:4654-4659 (1983) and Urlaub and Chasin PNAS USA 77:4216-4220 (1980), which are hereby incorporated by reference. The vector used in the methods of the present invention may be a viral vector, preferably a retroviral vector. Replication deficient adenoviruses are preferred. For example, a “single gene vector” in which the structural genes of a retrovirus are replaced by a single gene of interest, under the control of the viral regulatory sequences contained in the long terminal repeat, may be used, e.g. Moloney murine leukemia virus (MoMuIV), the Harvey murine sarcoma virus (HaMuSV), murine mammary tumor virus (MuMTV) and the murine myeloproliferative sarcoma virus (MuMPSV), and avian retroviruses such as reticuloendotheliosis virus (Rev) and Rous Sarcoma Virus (RSV), as described by Eglitis and Andersen, BioTechniques 6(7):608-614 (1988), which is hereby incorporated by reference.

Recombinant retroviral vectors into which multiple genes may be introduced may also be used according to the methods of the present invention. Vectors with internal promoters containing a cDNA under the regulation of an independent promoter, e.g. SAX vector derived from N2 vector with a selectable marker (noe.sup.R) into which the cDNA for human adenosine deaminase (hADA) has been inserted with its own regulatory sequences, the early promoter from SV40 virus (SV40), may be designed and used in accordance with the methods of the present invention by methods known in the art.

In mammalian host cells, a number of viral-based expression systems can be utilized. In cases where an adenovirus is used as an expression vector, the nucleotide sequence of interest (e.g., encoding a therapeutic capable agent) can be ligated to an adenovirus transcription or translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing the AQP1 gene product in infected hosts. (See e.g., Logan & Shenk, Proc. Natl. Acad. Sci. USA 8 1:3655-3659 (1984)).

Specific initiation signals can also be required for efficient translation of inserted therapeutic nucleotide sequences. These signals include the ATG initiation codon and adjacent sequences. In cases where an entire therapeutic gene or cDNA, including its own initiation codon and adjacent sequences, is inserted into the appropriate expression vector, no additional translational control signals can be needed. However, in cases where only a portion of the therapeutic coding sequence is inserted, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (See e.g., Bittner et al., Methods in Enzymol, 153:516-544 (1987)). Cells can be transfected with vectors or nucleic acid molecules (homologous recombination) comprising various inducible promoters for temporal regulation of gene expression.

Inducible expression systems are particularly useful in practicing the subject modulation methods. Non-limiting examples of inducible expression systems are listed below.

Common Eukaryotic Inducible Gene Expression Systems²

| | Induction | | | | | |
|---|---|---|---|---|---|---|
| | Ratio | | | | | |
| | (Fold | | | | | |
| System | Induction) | Advantages | | Disadvantages | | References |
| Heat Shock | 4-10 | 1. | Inducing agent readily | 1. | Many pleiotropic effects. | (Kothary et |
| | | | available and inexpensive; | 2. | Limited utility in mammalian | al., 1989) |
| | | 2. | Very fast induction kinetics | | systems. | |
| | | | (1 h.) | 3. | First reported to likely have low | |
| | | | | | leakiness; however, due to low | |
| | | | | | induction, more likely that | |
| | | | | | leakiness is high. | |
| Heavy metal | 5-10 | 1. | Fast induction kinetics (16 h). | 1. | Many pleiotropic effects (1992). | (Filmus et |
| ions | | 2. | High leakiness. | | | al.) |
| Interferon | 2-50 | 1. | Low leakiness | 1. | Slow induction kinetics (3-4 days) | (Kuhn et al., |
| | | | | 2. | Function dependent on cell type. | 1995) |
| | | | | 3. | Induction causes many pleiotropic | |
| | | | | | effects. | |
| FK506 | 0-1.5 | 1. | Very fast kinetics (16 h). | 1. | Limited usefulness; mostly in vitro | (Belshaw et |
| dimer | | 2. | Very low leakiness. | | to activate endrogenous signaling | al., 1996) |
| | | 3. | Few pleiotropic effects. | | pathways. | |
| Steroid | 0-200 | 1. | Fast induction kinetics (24 h). | 1. | High leakiness. | (Kuo et al., |
| | | | | 2. | Many pleiotropic effects. | 1994) |
| | | | | 3. | In vivo utility unknown. | |
| Gal4-Er | 0-100 | 1. | Very fast induction kinetics | 1. | In vivo utility unknown. | (Braselmann |
| | | | 1-2 h) | | | et al., 1993) |
| | | 2. | Low leakiness | | | |
| | | 3. | No pleiotropic effects. | | | |
| Progesterone | 10-50 | 1. | Fast induction kinetics | 1. | RU486 is unspecific for | (Wang et al., |
| antagomist/ | | | (10 h, in vitro). | | progesterone receptors. | 1994) |
| RU486 | | 2. | Low leakiness. | 2. | Not an ideal induction agent, | |
| | | 3. | Few pleiotropic effects in | | especially for potentially- | |
| | | | most subjects. | | pregnant/pregnant subjects. | |
| | | 4. | Receptor is endogenous to | 3. | Lower induction. | |
| | | | many subjects. | 4. | Receptors are not expressed by | |
| | | 5. | Inducer is readily available | | every cell types; limited tissue | |
| | | | and commonly used. | | distribution. | |
| Mutant | Unknown | 1. | Low leakiness | 1. | Slow induction kinetics (3-4 days). | (Zhang et al, |
| estrogen | most | 2. | No pleiotropic effects. | | | 1996) |
| receptor | subjects | | | | | |
| Eodysone | 0-10⁴ | 1. | Eodysone is not produces in | 1. | Low polypeptide production. | (No et al., |
| | | | most sublects. | 2. | Effects of eodsyone (or synthetic | 1996) |
| | | 2. | Fast induction kinetics (20 h). | | analogues) on mammalian | |
| | | 3. | Very low leakiness, | | physiology over time are unknown. | |
| | | 4. | No pleiotropic effects. | | | |
| Tetracycline | 1000-10⁶ | 1. | Tetracycline is not | 1. | Higher basal expression of | (Gossen et |
| | | | endogenous to subjects. | | operably-linked subeloned nucleic | al., 1995) |
| | | 2. | Tetracycline is readily available | | acid. | |
| | | | and commonly used. | 2. | Tetracycline is continuously | |
| | | 3. | Low leakiness. | | administered to a cell or subject (in | |
| | | 4. | Induction occurs upon the | | the version that is OFF in the | |
| | | | introduction of tetracycline- | | presence of tetracycline) | |
| | | | thus eliminating continuous | 3. | Tetracycline has undesirable side | |
| | | | tetracycline administration | | effects, such as squelching non- | |
| | | | (in the version that is OFF in | | specifically gene expression. | |
| | | | the absence of tetracycline). | 4. | In cultured cells, tetracycline is | |
| | | | | | difficult to wash out and this is | |
| | | | | | difficult to synchronize | |
| | | | | | polypeptide production. | |
| | | | | 5. | Unreliable; does not always work. | |
| Lac | 5-1000 | 1. | Fast induction kinetics (1214 | 1. | Induced expression is commonly | (Baim et al., |
| repressor- | | | 24 h) | | limited to only 40 to 50-fold | 1991) |
| based | | 2. | IPTG inducing agent is not | 2. | IPTG can exert cytotoxic success. | |
| | | | produced in subjects. | 3. | High leakiness. | |
| | | 3. | IPTG is easily available. | | | |

The subject agents designed to target NgRH1 may function as an agonist or antagonist so long as it mediates the effect in modulating BBB permeability. For example, where NgRH1 is necessary to maintain BBB integrity, then the agent (e.g., antibody specific for NgRH1) may function as an antagonist. Where signaling through NgRH1 breaks down the BBB (e.g., in pathological conditions e.g., MS), then the agent can act as an agonist.

In one embodiment, the altered BBB permeability is reversible or transient. A transient increase in BBB permeability avoids permanent damage to the BBB endothelium. This is particularly desirable for the application of diagnostic and/or therapeutic substances that are otherwise impermeable to BBB. Where desired, the agent increases the BBB permeability transiently for a period of time and to an extent that is sufficient to allow the agent across the BBB to yield the desired biological effect. For example, injection of an anti-NgRH1 antibody results in transient BBB permeability wherein the BBB impermeability is restored in less than about 2 hours. One may vary the amount of agents applied to control the duration of the period for which the BBB permeability is increased. Depending on the intended applications, the agent may increase the BBB permeability for about 5, 10, 20, 30, 40, 50, or 60 minutes. Alternatively, the agents may increase the BBB permeability for longer than 1, 2, 3, 4, or 5 hours. The degree of permeability can also be adjusted depending on the intended applications. The agent can be designed to slightly or dramatically increase the BBB permeability for a brief period of time, for example, in less than about 30, 20, 10, or 5 minutes. Conversely, agents can be applied to tighten the BBB, and hence decrease its permeability for a desired period of time and degree. By controlling dosage times/concentrations, BBB permeability modulation can be effectively permanent or for a desired period of time.

Identification of BBB Permeability Modulators

The present invention also provides a method of assessing whether a candidate agent modulates BBB permeability. The method comprises the steps of (a) exposing a subject's central nervous system to an indicator of said BBB permeability; (b) administering to said subject said candidate agent that targets NgRH1 cell surface receptor, wherein an increases or decrease in BBB permeability indicates that said candidate agent is capable of modulating BBB permeability.

BBB permeability can be tested by utilizing various indicators known in the art. (e.g., Olson et al. 1994; Saris et al. 1988; Rapoport 2000; Sternberger and Sternberger 1987; Ghabriel et al. 2000). For example, dyes, tracers or markers (e.g., sodium fluorescein or Evans blue) of a molecular weight greater that 180 Da are precluded from passage through an intact BBB. The assay can be conducted in experimental animals, including without limitation mice, rats, dogs, pigs, or monkeys.

Suitable indicators include any dye, marker, or tracer known in the art that is utilized to determine, visualize, measure, identify or quantify blood-brain barrier permeability. Non-limiting examples include, Evans Blue and sodium fluorescein. Examples of such indicators will be apparent to one of ordinary skill in the art, and include essentially any compound that is unable to traverse an intact BBB, but is capable of traversing a more permeable BBB, as well as capable of being identified, measured or quantified.

Indicators can be enzymes, tracers or markers utilized to determine BBB permeability changes, with non-limiting examples as follows:

| Enzyme | Functions observed | |
|---|---|---|
| Dopa-decarboxylase | Convert L-Dopa to dopamine | |
| Monoamine oxidase-B | Inactivates catecholamines (5-HT) | |
| Pseudocholinesterase | Deacetylates heroin to morphine | |
| Cytochrome P450 | O-Demethylates codeine to morphine | |
| UDP- | Metabolizes 1-naphthol | |
| Glucuronosyltransferase | | |
| Epoxide hydrolase | Reacts with epoxides | |
| | (Benzo[a]pyre 4,5-oxide) | |
| Renin | Angiotensinogen to Angiotensin I | |
| Dipeptidyl dipeptidase | Enkephalin metabolism | |
| ACE | Enkephalin, angiotensin I, neurotensin, and | |
| | bradykinin metabolism | |
| Aminopeptidase A | Metabolism of angiotensin | |
| Aminopeptidase M (N) | Opioid degradation (N-terminal Tyr) | |
| Glutamyl aminopeptidase | Convert angiotensin II to angiotensin III | |
| Enkephalinase* | Enkephalin, Endothelin, and bradyknin | |
| (neutral Endopeptidase | degradation | |
| 124. | 11) | |
| Endopeptidase* | Dynorphin, neurotensin, bradykinin, | |
| (Endopeptidase 24. | 15) | angiotensin II, and LHRH degradation |
| γGlutamyltranspeptidase | Convert leukotriene C4 to leukotriene D4 | |
| Alkaline phosphatase | purine and pyrimidine metabolism | |

*Enzymes in choroids plexus; ACE: angiotensin converting enzyme; LHRH: luteinizing hormone releasing hormone

Additional examples of dyes, tracers or markers include dextran, biotin, fibrinogen, albumin, blood globulin's using Coons's reaction, Texas Red conjugated dextran (70,000 da MW), Na(+)-fluorescein (MW 376) or fluorescein isothiocyanate (FITC) labelled dextran (MW 62,000 or 145,000), or FITC-labeled dextran of molecular mass 10,000 Da (FITC-dextran-10K).

Where desired, an altered BBB permeability can be correlated with an altered expression and/or activity of NgRH1 (including a splice variant thereof). An NgRH1 related gene or gene product can be determined by assaying for a difference in the mRNA levels of the corresponding genes before, during or after administration of the candidate agent. Alternatively, the differential expression of an NgRH1 related gene or gene product is determined by detecting a difference in the level of the encoded polypeptide or gene product

A wide variety of quantitative nucleic acid analyses are available in the art. They include but are not limited to quantitative PCR, DNA array hybridization and the like. A number of techniques for protein analysis based on the general principles outlined above are available in the art. They include but are not limited to radioimmunoassays, ELISA (enzyme linked immunoradiometric assays), “sandwich” immunoassays, immunoradiometric assays, in situ immunoassays (using e.g., colloidal gold, enzyme or radioisotope labels), western blot analysis, immunoprecipitation assays, immunofluorescent assays, and SDS-PAGE.

Antibodies that specifically recognize or bind to NgRH1 (including a splice variant thereof) are preferable for conducting the aforementioned protein analyses. These antibodies may be raised by conventional hybridoma technology or other methods available in the art.

The subject method may also be practised with the use of a transgenic animal. Transgenic animals can be broadly categorized into two types: “knockouts” and “knockins”. A “knockout” has an alteration in the target gene via the introduction of transgenic sequences that results in a decrease of function of the target gene, preferably such that target gene expression is insignificant or undetectable. A “knockin” is a transgenic animal having an alteration in a host cell genome that results in an augmented expression of a target gene, e.g., by introduction of an additional copy of the target gene, or by operatively inserting a regulatory sequence that provides for enhanced expression of an endogenous copy of the target gene. The knock-in or knock-out transgenic animals can be heterozygous or homozygous with respect to the target genes. The transgenic animals of the present invention can broadly be classified as Knockins which can over-express or under-express NgRH1.

In the present invention, transgenic animals are designed to provide a model system for determining, identifying and/or quantifying BBB permeability modulation. Such determinations can occur at any time during the animal's life span, including before or after BBB permeability disruption or modification. The transgenic model system can also be used for the development of biologically active agents that promote or increase BBB permeability. Furthermore, the model system can be utilized to assay whether a test agent restores the barrier or decreases permeability, e.g., post BBB ‘opening’ (i.e., increase permeability), such as, BBB opening resulting from trauma or disease. Moreover, cells can be isolated from the transgenic animals of the invention for further study or assays conducted in a cell-based or cell culture setting, including ex vivo techniques.

The animal models of the present invention encompass any non-human vertebrates that are amenable to procedures yielding a modified BBB permeability condition in the animal's nervous systems. Preferred model organisms include but are not limited to mammals, primates, and rodents. Non-limiting examples of the preferred models are rats, mice, guinea pigs, cats, dogs, rabbits, pigs, chimpanzees, and monkeys. The test animals can be wildtype or transgenic.

In some aspect of the invention, the transgenic animal is NgRH1 deficient. In yet other aspects of the invention, the NgRH1 deficient animal is engineered to encompss additional genotype/phenotypic backgrounds. In one embodiment the additional background is deficient in one or more tight junction proteins selected from occluding, claudin 7, claudin 10 and claudin 11. In yet other embodiments, the animal can be deficient for a protein selected from claudin 2, claudin 5, claudin 6, claudin 12, clauding 15 and claudin 19.

Advances in technologies for embryo micromanipulation now permit introduction of heterologous DNA into fertilized mammalian ova as well. For instance, totipotent or pluripotent stem cells can be transformed by microinjection, calcium phosphate mediated precipitation, liposome fusion, retroviral infection or other means. The transformed cells are then introduced into the embryo, and the embryo will then develop into a transgenic animal. In a preferred embodiment, developing embryos are infected with a viral vector containing a desired transgene so that the transgenic animals expressing the transgene can be produced from the infected embryo. In another preferred embodiment, a desired transgene is co-injected into the pronucleus or cytoplasm of the embryo, preferably at the single cell stage, and the embryo is allowed to develop into a mature transgenic animal. These and other variant methods for generating transgenic animals are well established in the art and hence are not detailed herein. See, for example, U.S. Pat. Nos. 5,175,385 and 5,175,384.

Delivery of Therapeutic Agents to CNS

The present invention also features method of delivering a therapeutic agent to a central nervous system (CNS) of a subject. The method comprises administering the therapeutic agent to the CNS prior to, concurrent with, or subsequent to, increasing BBB permeability as a result of modulating NgRH1 cell surface receptor activity and/or expression level. Such method can be employed in treatment of any CNS diseases and especially BBB related disorders (e.g., where the barrier integrity is compromised).

Any of the agents described herein that target the NgRH1 (including splice variants) can be employed in the subject methods. In some embodiments, the agent administered is an antibody targeting NgRH1 (including splice variants), to modulate BBB permeability.

By way of illustration where an antibody is employed, the antibody targets NgRH1 and increases BBB permeability in the subject. For example, the subject is suffering or likely to suffer from a BBB-related disorder, where increased permeability will provide therapeutic capabilities. Such therapeutic capabilities can include delivery across the BBB of therapeutic agents to ameliorate disease. Many conditions specific to the CNS are difficult to treat because therapeutic agents are precluded from traversing across the BBB. Thus, administration of an antibody in such cases will down-regulate NgRH1 function or activity resulting in increased permeability, which in turn allows certain therapeutics to be delivered to the CNS.

The delivery of the therapeutic agent can be concurrent with, or subsequent to, increasing BBB permeability as a result of modulating NgRH1 cell surface receptor activity and/or expression level.

Such method can be employed in treatment of any CNS diseases and especially. BBB related disorders (e.g., where the barrier integrity is compromised). In another embodiment, in a subject suffering from a BBB-related disorder (e.g., ‘open’ BBB), the antibody is administered to effectually restore or maintain the barrier. For example, the antibody can be administered to a subject suffering from detrimental effects of albumin (or some any protein that does not traverse an intact BBB) traversing the BBB. In such a subject, the antibody targeting NgRH1 up-regulates NgRH1 function thus decreasing permeability. In effect, the down-regulation of NgRH1 equates to restoration of the BBB. Methods of producing antibodies for use in one or methods of the present invention, including pharmaceutically acceptable forms of such antibodies, are known to one of ordinary skill in the art. (e.g., U.S. Pat. Nos. 5,585,097; 5,846,534; 6,706,265; 6,982,321; 6,491,916; 5,885,573; or WO 04/043386; EP 1098909; or US Patent Publication NO. 2003/0216551; 2005/0037000; 2005/0064514; or 2005/0054832).

Examples of BBB-related conditions/disorders include Amyotropic Lateral Sclerosis (ALS), Multiple Sclerosis (MS), Immune Dysfunction Muscular Central Nervous System Breakdown, Muscular Dystrophy (MD), Alzheimer's disease, Pakinson's disease, Huntington's disease, Brain Ischemia, Cerebral Palsy, Corticobasal Ganglionic Degeneration, Creutzfeldt-Jakob Syndrome, Dandy-Walker Syndrome, Dementia, Vascular Encephalitis, Encephalomyelitis, Epilepsy, Essential Tremor, Kuru-Landau-Kleffner Syndrome, Lewy Body Disease, Machado-Joseph Disease, Meige syndrome, Migraine Disorders, poliomyelitis, Multiple System Atrophy, Meningitis, Drager Syndrome, Tourette Syndrome, Hallervorden-Spatz Syndrome, Hydrocephalus, Oliovopontocerebellar atrophies, Supranucleal Palsy, or Syringomyelia.

In one embodiment, a compound (which may be a peptide or other molecule that is capable of binding to the antibody) is reversibly bound to the antibody binding or combining site of the antibody that is to be administered to a person. The compound occupies the binding site of the antibody for the antigen and thereby inhibits binding of the antibody to the antigen. Since the compound is reversibly bound to the antibody binding site and is selected to have a limited reduction in antibody binding, the antibody is capable of binding to the antigen against which the antibody is directed. As such, the combination and concentration of the antibody and the compound will provide one sensitive mechanism for modulating NgRH1 function thus BBB permeability.

Dosage levels of such agents can be in the order of from about 0.1 mg to about 140 mg per kilogram of body weight, 0.01 to 100 mg per kilogram of body, useful in the treatment of the BBB-related conditions (about 0.5 mg to about 7 g per patient or subject per day). The amount of agent can be combined with the carrier materials to produce a single dosage form, which varies depending upon the host treated and the particular mode of administration. Dosage unit forms generally contain between from about 1 mg to about 500 mg of an active ingredient. It is understood that the specific dose level for any particular patient or subject depends upon a variety of factors including the activity of the specific compound employed, the age, body weight, general health, sex, diet, time of administration, route of administration, and rate of excretion, drug combination and the severity of the particular disease undergoing therapy. The dosage regimens described herein are equally applicable to nucleic acid, antibody, ligand, peptide or protein molecules targeting NgRH1.

In one aspect, the invention is directed to a method for modulating BBB permeability comprising, administering to a subject at least one NgRH1-specific biologically active agent or binding fragment thereof, which is administered at a dose of between about 20 mg/kg to 40 mg/kg in one or more separate doses. In some embodiments, the biologically active agent is an antibody and/or a ligand. In one embodiment, the antibody is SMI71. In one embodiment, the ligand is MAG.

In one embodiment, the at least one NgRH1-specific antibody and/or ligand is administered at a dose of about 20 mg/kg. Furthermore, the antibody is administered in one or more doses. Where desired, NgRH1 antibody is administered at a dose sufficient to maintain a serum concentration of anti-NgRH1 antibody at a level of about 20 μg/ml during treatment.

In one preferred embodiment as applicable to one or methods of the present invention, at least one biologically active agent targeting NgRH1 is administered in doses on separate occasions, where the number of doses administered is at least 1, 2, 3, 4, 5, 6, 7, 8, 9, 10, 11, 12, 13, 14, 15, 16, 17, 18, 19, or 20 doses and/or the number of occasions on which doses are administered is at least 1, 2, 3, 4, 5, 6, 7, 8, 9, 10, 11, 12, 13, 14, 15, 16, 17, 18, 19, or 20 different time points over a period of weeks, months, years where needed, throughout the lifetime of a subject. In such embodiments, the biologically active agent is an antibody or ligand (e.g., SMI71 or MAG).

In another aspect, the invention is directed to one or more methods of the invention, wherein at least one NgRH1 biologically active agent is administered at a dose sufficient to achieve about 85% saturation of NgRH1 sites on vascular endothelial cells in a subject during treatment. In some embodiments, the saturation level is between selected from a level of about at least 50, 55, 60, 65, 70, 75, 80, 85, 90, or 95%.

Therapeutic agents can be delivered as a therapeutic or as a prohpylactic (e.g., inhibiting or preventing onset of neurodegenerative diseases). By therapeutic benefit is meant eradication or amelioration of the underlying disorder being treated. For prophylactic benefit, the agents may be administered to a patient at risk of developing a disease or to a patient reporting one or more of the physiological symptoms of such a disease, even though a diagnosis may not have yet been made. Alternatively, prophylactic administration may be applied to avoid the onset of the physiological symptoms of the underlying disorder, particularly if the symptom manifests cyclically. In this latter embodiment, the therapy is prophylactic with respect to the associated physiological symptoms instead of the underlying indication. The actual amount effective for a particular application will depend, inter alia, on the condition being treated and the route of administration.

In one aspect of the invention, the therapeutic capable agents may be selected from a group consisting of immunosuppressants, anti-inflammatories, anti-proliferatives, anti-migratory agents, anti-fibrotic agents, proapoptotics, calcium channel blockers, anti-neoplastics, antibodies, anti-thrombotic agents, anti-platelet agents, IIbIIIIa agents, antiviral agents, and a combination thereof. Specific examples of therapeutic capable agent include: mycophenolic acid, mycophenolate mofetil, mizoribine, methylprednisolone, dexamethasone, Certican, rapamycin, Triptolide, Methotrexate, Benidipine, Ascomycin, Wortmannin, LY294002, Camptothecin, Topotecan, hydroxyurea, Tacrolimus (FK 506), cyclophosphamide, cyclosporine, daclizumab, azathioprine, prednisone, Gemcitabine, derivatives, pharmaceutical salts and combinations thereof.

Additional examples of therapeutic capable agent comprise at least one compound selected from the group consisting of anti-cancer agents; chemotherapeutic agents; thrombolytics; vasodilators; antimicrobials or antibiotics; antimitotics; growth factor antagonists; free radical scavengers; biologic agents; radio therapeutic agents; radiopaque agents; radiolabelled agents; anti-coagulants such as heparin and its derivatives; anti-angiogenesis drugs such as Thalidomide; angiogenesis drugs; PDGF-B and/or EGF inhibitors; anti-inflammatories including psoriasis drugs; riboflavin; tiazofurin; zafurin; anti-platelet agents including cyclooxygenase inhibitors such as acetylsalicylic acid, ADP inhibitors such as clopidogrel (e.g., Plavix) and ticlopdipine (e.g., ticlid), hosphodiesterase I11 inhibitors such as cilostazol (e.g., Pletal)g, lycoprotein II/IIIIa agents such as abciximab (e.g., Rheopro™); eptifibatide (e.g., Integrilin), and adenosine reuptake inhibitors such as dipyridmoles; healing and/or promoting agents including anti-oxidants, nitrogen oxide donors; antiemetics; antinauseants; tripdiolide, diterpenes, triterpenes, diterpene epoxides, diterpenoid epoxide, triepoxides, or tripterygium wifordii hook F(TWHF), SDZ-RAD, RAD, RAD666, or 40-0-(2-hydroxy)ethyl-rapamycin, derivatives, pharmaceutical salts and combinations thereof.

Anti-tumor or anti-cancer agents can also be delivered utilizing one or more methods of the invention. An anti-tumor or anti-cancer therapeutic capable agent is a molecule which decreases or prevents a further increase in growth of a tumor and includes anti-cancer agents such as Acivicin; Aclarubicin; Acodazole Hydrochloride; Acronine; Adriamycin; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-1b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Tecogalan Sodium; Tegafur; Teloxantrone Hydrochloride; Temoporfin; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride, and Taxol.

In another aspect of the invention, the therapeutic capable agent is a bioactive protein or peptide. Examples of such bioactive protein or peptides include a cell modulating peptide, a chemotactic peptide, an anticoagulant peptide, an antithrombotic peptide, an anti-tumor peptide, an anti-infectious peptide, a growth potentiating peptide, and an anti-inflammatory peptide. Examples of proteins include antibodies, enzymes, steroids, growth hormone and growth hormone-releasing hormone, gonadotropin-releasing hormone, and its agonist and antagonist analogues, somatostatin and its analogues, gonadotropins such as luteinizing hormone and follicle-stimulating hormone, peptide T, thyrocalcitonin, parathyroid hormone, glucagon, vasopressin, oxytocin, angiotensin I and II, bradykinin, kallidin, adrenocorticotropic hormone, thyroid stimulating hormone, insulin, glucagon and the numerous analogues and congeners of the foregoing molecules. The therapeutic agents may be selected from insulin, antigens selected from the group consisting of MMR (mumps, measles and rubella) vaccine, typhoid vaccine, hepatitis A vaccine, hepatitis B vaccine, herpes simplex virus, bacterial toxoids, cholera toxin B-subunit, influenza vaccine virus, bordetela pertussis virus, vaccinia virus, adenovirus, canary pox, polio vaccine virus, plasmodium falciparum, bacillus calmette geurin (BCG), klebsiella pneumoniae, HIV envelop glycoproteins and cytokins and other agents selected from the group consisting of bovine somatropine (sometimes referred to as BST), estrogens, androgens, insulin growth factors (sometimes referred to as IGF), interleukin I, interleukin II and cytokins. Three such cytokins are interferon-α, interferon-β and tuftsin.

In some aspects of the invention, the BBB permeability is modulated by one or more methods described herein above, so as to deliver an antibiotic, or an anti-infectious therapeutic capable agent. Such anti-infectious agents reduce the activity of or kills a microorganism and includes Aztreonam; Chlorhexidine Gluconate; Imidurea; Lycetamine; Nibroxane; Pirazmonam Sodium; Propionic Acid; Pyrithione Sodium; Sanguinarium Chloride; Tigemonam Dicholine; Acedapsone; Acetosulfone Sodium; Alamecin; Alexidine; Amdinocillin; Amdinocillin Pivoxil; Amicycline; Amifloxacin; Amifloxacin Mesylate; Amikacin; Amikacin Sulfate; Aminosalicylic acid; Aminosalicylate sodium; Amoxicillin; Amphomycin; Ampicillin; Ampicillin Sodium; Apalcillin Sodium; Apramycin; Aspartocin; Astromicin Sulfate; Avilamycin; Avoparcin; Azithromycin; Azlocillin; Azlocillin Sodium; Bacampicillin Hydrochloride; Bacitracin; Bacitracin Methylene Disalicylate; Bacitracin Zinc; Bambermycins; Benzoylpas Calcium; Berythromycin; Betamicin Sulfate; Biapenem; Biniramycin; Biphenamine Hydrochloride; Bispyrithione Magsulfex; Butikacin; Butirosin Sulfate; Capreomycin Sulfate; Carbadox; Carbenicillin Disodium; Carbenicillin Indanyl Sodium; Carbenicillin Phenyl Sodium; Carbenicillin Potassium; Carumonam Sodium; Cefaclor; Cefadroxil; Cefamandole; Cefamandole Nafate; Cefamandole Sodium; Cefaparole; Cefatrizine; Cefazaflur Sodium; Cefazolin; Cefazolin Sodium; Cefbuperazone; Cefdinir; Cefepime; Cefepime Hydrochloride; Cefetecol; Cefixime; Cefmenoxime Hydrochloride; Cefmetazole; Cefmetazole Sodium; Cefonicid Monosodium; Cefonicid Sodium; Cefoperazone Sodium; Ceforanide; Cefotaxime Sodium; Cefotetan; Cefotetan Disodium; Cefotiam Hydrochloride; Cefoxitin; Cefoxitin Sodium; Cefpimizole; Cefpimizole Sodium; Cefpiramide; Cefpiramide Sodium; Cefpirome Sulfate; Cefpodoxime Proxetil; Cefprozil; Cefroxadine; Cefsulodin Sodium; Ceftazidime; Ceftibuten; Ceftizoxime Sodium; Ceftriaxone Sodium; Cefuroxime; Cefuroxime Axetil; Cefuroxime Pivoxetil; Cefuroxime Sodium; Cephacetrile Sodium; Cephalexin; Cephalexin Hydrochloride; Cephaloglycin; Cephaloridine; Cephalothin Sodium; Cephapirin Sodium; Cephradine; Cetocycline Hydrochloride; Cetophenicol; Chloramphenicol; Chloramphenicol Palmitate; Chloramphenicol Pantothenate Complex; Chloramphenicol Sodium Succinate; Chlorhexidine Phosphanilate; Chloroxylenol; Chlortetracycline Bisulfate; Chlortetracycline Hydrochloride; Cinoxacin; Ciprofloxacin; Ciprofloxacin Hydrochloride; Cirolemycin; Clarithromycin; Clinafloxacin Hydrochloride; Clindamycin; Clindamycin Hydrochloride; Clindamycin Palmitate Hydrochloride; Clindamycin Phosphate; Clofazimine; Cloxacillin Benzathine; Cloxacillin Sodium; Cloxyquin; Colistimethate Sodium; Colistin Sulfate; Coumermycin; Coumermycin Sodium; Cyclacillin; Cycloserine; Dalfopristin; Dapsone; Daptomycin; Demeclocycline; Demeclocycline Hydrochloride; Demecycline; Denofungin; Diaveridine; Dicloxacillin; Dicloxacillin Sodium; Dihydrostreptomycin Sulfate; Dipyrithione; Dirithromycin; Doxycycline; Doxycycline Calcium; Doxycycline Fosfatex; Doxycycline Hyclate; Droxacin Sodium; Enoxacin; Epicillin; Epitetracycline Hydrochloride; Erythromycin; Erythromycin Acistrate; Erythromycin Estolate; Erythromycin Ethylsuccinate; Erythromycin Gluceptate; Erythromycin Lactobionate; Erythromycin Propionate; Erythromycin Stearate; Ethambutol Hydrochloride; Ethionamide; Fleroxacin; Floxacillin; Fludalanine; Flumequine; Fosfomycin; Fosfomycin Tromethamine; Fumoxicillin; Furazolium Chloride; Furazolium Tartrate; Fusidate Sodium; Fusidic Acid; Gentamicin Sulfate; Gloximonam; Gramicidin; Haloprogin; Hetacillin; Hetacillin Potassium; Hexedine; Ibafloxacin; Imipenem; Isoconazole; Isepamicin; Isoniazid; Josamycin; Kanamycin Sulfate; Kitasamycin; Levofuraltadone; Levopropylcillin Potassium; Lexithromycin; Lincomycin; Lincomycin Hydrochloride; Lomefloxacin; Lomefloxacin Hydrochloride; Lomefloxacin Mesylate; Loracarbef; Mafenide; Meclocycline; Meclocycline Sulfosalicylate; Megalomicin Potassium Phosphate; Mequidox; Meropenem; Methacycline; Methacycline Hydrochloride; Methenamine; Methenamine Hippurate; Methenamine Mandelate; Methicillin Sodium; Metioprim; Metronidazole Hydrochloride; Metronidazole Phosphate; Mezlocillin; Mezlocillin Sodium; Minocycline; Minocycline Hydrochloride; Mirincamycin Hydrochloride; Monensin; Monensin Sodium; Nafcillin Sodium; Nalidixate Sodium; Nalidixic Acid; Natamycin; Nebramycin; Neomycin Palmitate; Neomycin Sulfate; Neomycin Undecylenate; Netilmicin Sulfate; Neutramycin; Nifuradene; Nifuraldezone; Nifuratel; Nifuratrone; Nifurdazil; Nifurimide; Nifurpirinol; Nifurquinazol; Nifurthiazole; Nitrocycline; Nitrofurantoin; Nitromide; Norfloxacin; Novobiocin Sodium; Ofloxacin; Ormetoprim; Oxacillin Sodium; Oximonam; Oximonam Sodium; Oxolinic Acid; Oxytetracycline; Oxytetracycline Calcium; Oxytetracycline Hydrochloride; Paldimycin; Parachlorophenol; Paulomycin; Pefloxacin; Pefloxacin Mesylate; Penamecillin; Penicillin G Benzathine; Penicillin G Potassium; Penicillin G Procaine; Penicillin G Sodium; Penicillin V; Penicillin V Benzathine; Penicillin V Hydrabamine; Penicillin V Potassium; Pentizidone Sodium; Phenyl Aminosalicylate; Piperacillin Sodium; Pirbenicillin Sodium; Piridicillin Sodium; Pirlimycin Hydrochloride; Pivampicillin Hydrochloride; Pivampicillin Pamoate; Pivampicillin Probenate; Polymyxin B Sulfate; Porfiromycin; Propikacin; Pyrazinamide; Pyrithione Zinc; Quindecamine Acetate; Quinupristin; Racephenicol; Ramoplanin; Ranimycin; Relomycin; Repromicin; Rifabutin; Rifametane; Rifamexil; Rifamide; Rifampin; Rifapentine; Rifaximin; Rolitetracycline; Rolitetracycline Nitrate; Rosaramicin; Rosaramicin Butyrate; Rosaramicin Propionate; Rosaramicin Sodium Phosphate; Rosaramicin Stearate; Rosoxacin; Roxarsone; Roxithromycin; Sancycline; Sanfetrinem Sodium; Sarmoxicillin; Sarpicillin; Scopafungin; Sisomicin; Sisomicin Sulfate; Sparfloxacin; Spectinomycin Hydrochloride; Spiramycin; Stallimycin Hydrochloride; Steffimycin; Streptomycin Sulfate; Streptonicozid; Sulfabenz; Sulfabenzamide; Sulfacetamide; Sulfacetamide Sodium; Sulfacytine; Sulfadiazine; Sulfadiazine Sodium; Sulfadoxine; Sulfalene; Sulfamerazine; Sulfameter; Sulfamethazine; Sulfamethizole; Sulfamethoxazole; Sulfamonomethoxine; Sulfamoxole; Sulfanilate Zinc; Sulfanitran; Sulfasalazine; Sulfasomizole; Sulfathiazole; Sulfazamet; Sulfisoxazole; Sulfisoxazole Acetyl; Sulfisoxazole Diolamine; Sulfomyxin; Sulopenem; Sultamicillin; Suncillin Sodium; Talampicillin Hydrochloride; Teicoplanin; Temafloxacin Hydrochloride; Temocillin; Tetracycline; Tetracycline Hydrochloride Tetracycline Phosphate Complex; Tetroxoprim; Thiamphenicol; Thiphencillin Potassium; Ticarcillin Cresyl Sodium; Ticarcillin Disodium; Ticarcillin Monosodium; Ticlatone; Tiodonium Chloride; Tobramycin; Tobramycin Sulfate; Tosufloxacin; Trimethoprim; Trimethoprim Sulfate; Trisulfapyrimidines; Troleandomycin; Trospectomycin Sulfate; Tyrothricin; Vancomycin; Vancomycin Hydrochloride; Virginiamycin; Zorbamycin; Difloxacin Hydrochloride; Lauryl Isoquinolinium Bromide; Moxalactam Disodium; Omidazole; Pentisomicin; and Sarafloxacin Hydrochloride, as well as derivations, and combinations thereof.

In some aspects of the invention, the BBB permeability is modulated to deliver an anti-inflammatory therapeutic capable agent. Such an anti-inflammatory agent reduces an inflammatory response and includes steroidal and non-steroidal compounds; Alclofenac; Aldlometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; s Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; Zomepirac Sodium.

Additional nonsteroidal anti-inflammatory agents that may be used include, but are not limited to, aspirin, diclofenac, flurbiprofen, ibuprofen, ketorolac, naproxen, and suprofen. In a further variation, the antiinflammatory agent is a steroidal anti-inflammatory agent.

In some aspects of the invention, the BBB permeability is modulated to deliver a therapeutic capable agent that is an anticoagulant. Such an anticoagulant agent is a molecule that prevents clotting of blood and includes but is not limited to Ancrod; Anticoagulant Citrate Dextrose Solution; Anticoagulant Citrate Phosphate Dextrose Adenine Solution; Anticoagulant Citrate Phosphate Dextrose Solution; Anticoagulant Heparin Solution; Anticoagulant Sodium Citrate Solution; Ardeparin Sodium; Bivalirudin; Bromindione; Dalteparin Sodium; Desirudin; Dicumarol; Heparin Calcium; Heparin Sodium; Lyapolate Sodium; Nafamostat Mesylate; Phenprocoumon; Tinzaparin Sodium; Warfarin Sodium.

In some aspects of the invention, the BBB permeability is modulated to deliver a therapeutic capable agent that is antithrombotic. An antithrombotic molecule as used herein is a molecule that prevents formation of a thrombus and includes but is not limited to Anagrelide Hydrochloride; Bivalirudin; Dalteparin Sodium; Danaparoid Sodium; Dazoxiben Hydrochloride; Efegatran Sulfate; Enoxaparin Sodium; Ifetroban; Ifetroban Sodium; Tinzaparin Sodium; Trifenagrel.

In some aspects of the invention, imaging agents such as radioisotopes and fluorescent agents are delivered through the altered BBB. There agents are particularly useful for imaging the CNS or specific region of CNS. In addition, radioisotopes can be used for the treatment of cancer and other pathological conditions, as described, e.g., in Harbert, “Nuclear Medicine Therapy”, New York, Thieme Medical Publishers, 1987, pp. 1-340. In some embodiments the radio isotopes include but are not limited to isotopes and salts of isotopes with short half life: such as Y-90, P-32, 1-131, Au 198.

It is also well known that radioisotopes, drugs, and toxins can be conjugated to antibodies or antibody fragments which specifically bind to markers which are produced by or associated with particular cells, and that such antibody conjugates can be used to target the radioisotopes, drugs or toxins to tumor sites to enhance their therapeutic efficacy and minimize side effects. Examples of these agents and methods are reviewed in Wawrzynczak and Thorpe (in Introduction to the Cellular and Molecular Biology of Cancer, L. M. Franks and N. M. Teich, eds, Chapter 18, pp. 378-410, Oxford University Press, Oxford, 1986), in Immunoconjugates. Antibody Conjugates in Radioimaging and Therapy of Cancer (C.-W. Vogel, ed., 3-300, Oxford University Press, New York, 1987), in Dillman, R. O. (CRC Critical Reviews in Oncology/Hematology 1:357, CRC Press, Inc., 1984), in Pastan et al. (Cell 47:641, 1986), in Vitetta et al. (Science 238:1098-1104, 1987) and in Brady et al. (Int. J. Rad. Oncol. Biol. Phys. 13:1535-1544, 1987). Other examples of the use of immunoconjugates for cancer and other forms of therapy have been disclosed, inter alia, in Goldenberg, U.S. Pat. Nos. 4,331,647, 4,348,376, 4,361,544, 4,468,457, 4,444,744, 4,460,459, 4,460,561 and 4,624,846, and in Rowland, U.S. Pat. No. 4,046,722, Rodwell et al., U.S. Pat. No. 4,671,958, and Shih et al., U.S. Pat. No. 4,699,784, the disclosures of all of which are incorporated herein in their entireties by reference.

Angiogenic or anti-angiogenesis factors can also be delivered to treat a condition of the brain when needed. Angiogenesis, the growth of new blood vessels in tissue, has been the subject of increased study in recent years. Such blood vessel growth to provide new supplies of oxygenated blood to a region of tissue has the potential to remedy a variety of tissue and muscular ailments, particularly ischemia. Primarily, study has focused on perfecting angiogenic factors such as human growth factors produced from genetic engineering techniques. It has been reported that injection of such a growth factor into myocardial tissue initiates angiogenesis at that site, which is exhibited by a new dense capillary network within the tissue. Schumacher et al., “Induction of Neo-Angiogenesis in Ischemic Myocardium by Human Growth Factors”, Circulation, 1998; 97:645-650. Angiogenic factors include but are not limited to: VEGF, Hypoxia inducible factor (HIF), fibroblast growth factor (FGF), HO-1, SOD, NOSII, NOSIII, placental growth factor (PLGF), TGF.beta., angiopoietin-1, bFGF, and macrophage chemoattractant protein-1 (MCP-1), as well as functional derivatives or combinations thereof.

The treatment duration and regimen can vary depending on the particular condition and subject that is to be treated. For instance, a therapeutic agent can be administered by the subject method over at least 1, 7, 14, 30, 60, 90 days, or a period of months, years, or even throughout the lifetime of a subject.

Pharmaceutical Compositions of the Present Invention

The one or more methods of the invention disclosed herein can be utilized to select a biologically active agent that can subsequently be implemented in treatment of demyelination. The selected biologically active agents effective to modulate remyelination may be used for the preparation of medicaments for treating neuronal demyelination disorders. In one aspect, an identified/selected biologically active agent of this invention can be administered to treat neuronal demyelination inflicted by pathogens such as bacteria and viruses. In another aspect, the selected agent can be used to treat neuronal demyelination caused by toxic substances or accumulation of toxic metabolites in the body as in, e.g., central pontine myelinolysis and vitamin deficiencies. In yet another aspect, the agent can be used to treat demyelination caused by physical injury, such as spinal cord injury. In still yet another aspect, the agent can be administered to treat demyelination manifested in disorders having genetic attributes, genetic disorders including but not limited to leukodystrophies, adrenoleukodystrophy, degenerative multi-system atrophy, Binswanger encephalopathy, tumors in the central nervous system, and multiple sclerosis.

Various delivery systems are known and can be used to administer a biologically active agent of the invention, e.g., encapsulation in liposomes, microparticles, microcapsules, expression by recombinant cells, receptor-mediated endocytosis (see, e.g., Wu and Wu, (1987), J. Biol. Chem. 262:4429-4432), construction of a therapeutic nucleic acid as part of a retroviral or other vector, etc. Methods of delivery include but are not limited to intra-arterial, intramuscular, intravenous, intranasal, and oral routes. In a specific embodiment, it may be desirable to administer the pharmaceutical compositions of the invention locally to the area in need of treatment; this may be achieved by, for example, and not by way of limitation, local infusion during surgery, by injection, or by means of a catheter. In certain embodiment, the agents are delivered to a subject's nerve systems, preferably the central nervous system. In another embodiment, the agents are administered to neuronal tissues undergoing remyelination.

Administration of the selected agent can be effected in one dose, continuously or intermittently throughout the course of treatment. Methods of determining the most effective means and dosage of administration are well known to those of skill in the art and will vary with the composition used for therapy, the purpose of the therapy, the target cell being treated, and the subject being treated. Single or multiple administrations can be carried out with the dose level and pattern being selected by the treating physician.

The preparation of pharmaceutical compositions of this invention is conducted in accordance with generally accepted procedures for the preparation of pharmaceutical preparations. See, for example, Remington's Pharmaceutical Sciences 18th Edition (1990), E. W. Martin ed., Mack Publishing Co., PA. Depending on the intended use and mode of administration, it may be desirable to process the active ingredient further in the preparation of pharmaceutical compositions. Appropriate processing may include mixing with appropriate non-toxic and non-interfering components, sterilizing, dividing into dose units, and enclosing in a delivery device.

Pharmaceutical compositions for oral, intranasal, or topical administration can be supplied in solid, semi-solid or liquid forms, including tablets, capsules, powders, liquids, and suspensions. Compositions for injection can be supplied as liquid solutions or suspensions, as emulsions, or as solid forms suitable for dissolution or suspension in liquid prior to injection. For administration via the respiratory tract, a preferred composition is one that provides a solid, powder, or aerosol when used with an appropriate aerosolizer device.

Liquid pharmaceutically acceptable compositions can, for example, be prepared by dissolving or dispersing a polypeptide embodied herein in a liquid excipient, such as water, saline, aqueous dextrose, glycerol, or ethanol. The composition can also contain other medicinal agents, pharmaceutical agents, adjuvants, carriers, and auxiliary substances such as wetting or emulsifying agents, and pH buffering agents.

### EXAMPLES

### Example 1

Tracer permeability studies: Rats were anesthetized with a ketamine/xylazine cocktail. The thoracic cavity was dissected open and the right atrium was cut open with small dissecting scissors. Biotin (1-2 mg/ml in DPBS) was perfused into the left ventricle for 15 minutes using a dynamax peristaltic pump for 10 minutes, followed by 10-15 minutes of 4% paraformaldehyde. The fixed rat brain/tissues were then incubated in 4% paraformaldehyde overnight at 4 degrees before being sunk in 30% sucrose. The brain and tissue was then frozen in a 2:1 30% sucrose to OCT mixture and 12-16 um cryosections were made using a cryostat. Sections were rehydrated in PBS, and then blocked with 50% goat serum before incubation with a 1:500 streptavidin alexa-488. Avidin-biotin complexes were then visualized by fluorescent microscopy. Permeability was also assessed utilizing a 10 kD rhodamine-dextran (0.5 mg/ml in DPBS) instead of biotin. In this case the dextran was visualized directly after sectioning.

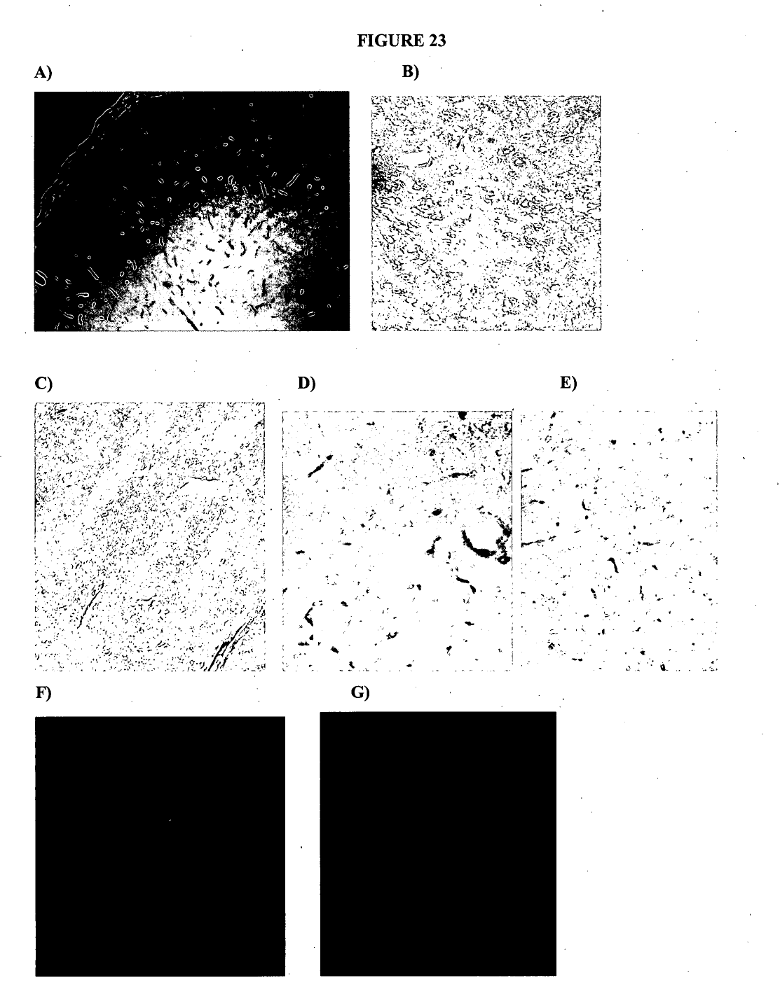

Utilizing immunohistochemistry it was demonstrated EBA immunoreactivity can be visualized specifically in blood vessels throughout the adult rat central nervous system, but not in the peripheral tissues such as the liver, lung or muscle (FIG. 23). EBA reactivity is not visualized during early postnatal development, but is first observed in a subset of CNS vessels at p17, and then throughout all CNS vessels by p20 (FIG. 23; C-D). This is in contrast with other known BBB markers including occludin, p-glycoprotein, glut-1 and the transferrin receptor, which are expressed by CNS endothelial cells starting in embryogenesis an persist throughout postnatal life.

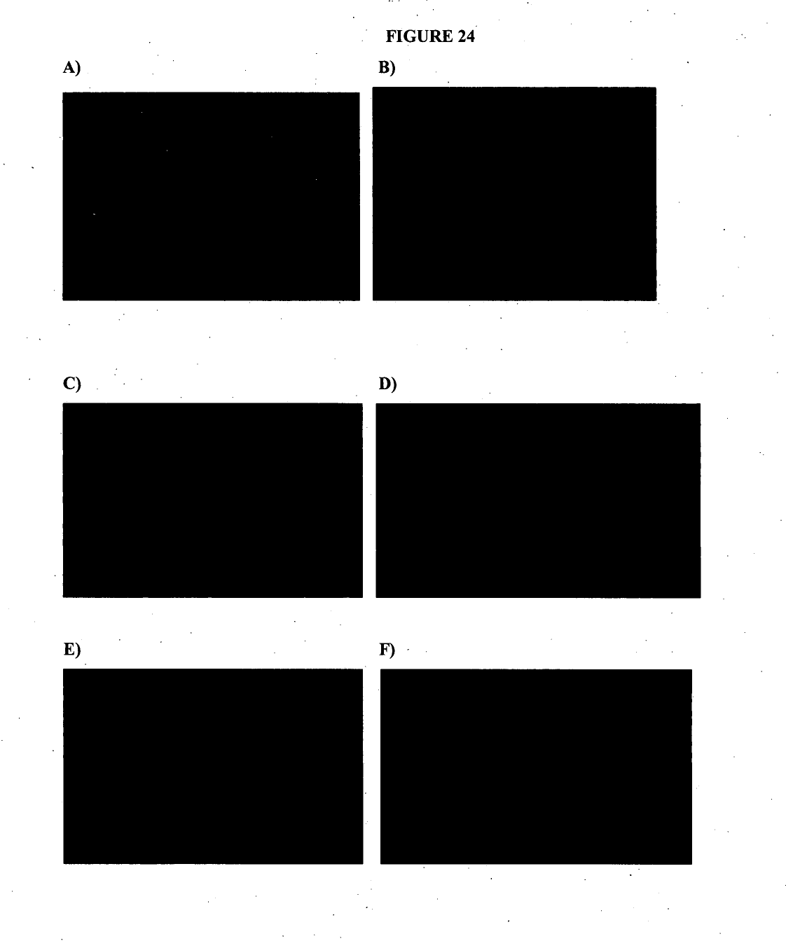

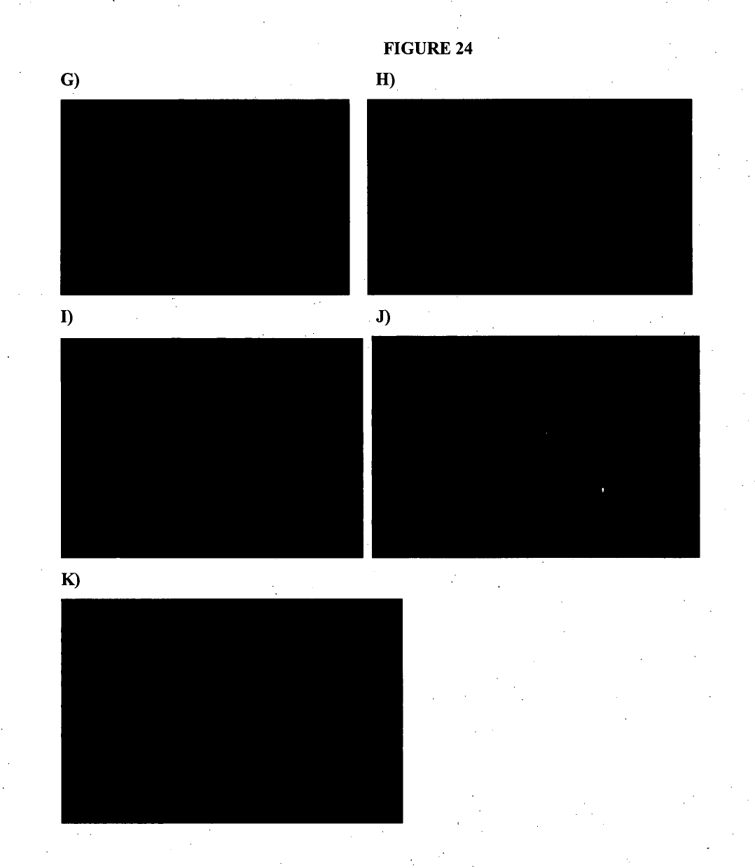

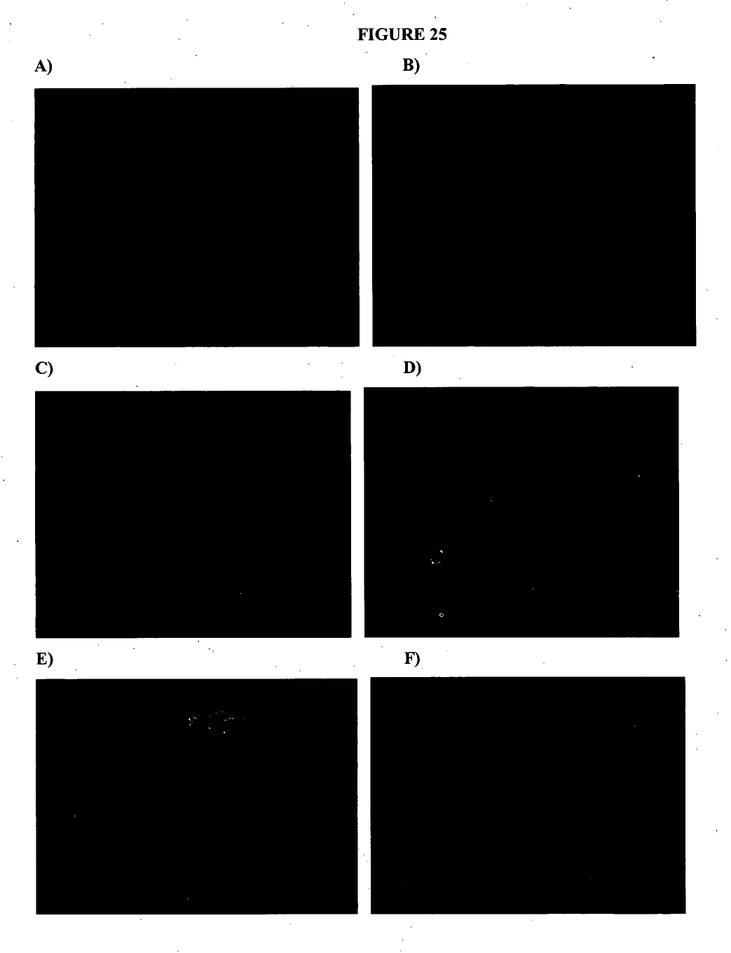

The permeability of the BBB is determined by perfusing rats with molecular tracers and visualizing whether these tracers are able to diffuse into the brain parenchyma. It was demonstrated that after birth and throughout postnatal development and adulthood, the BBB is impermeable to biotin (MW ˜250 Dalton 1 mg/ml) or tetramethyl-rhodamine dextran (˜10 kD, 2 mg/ml) indicating that the BBB forms prior to postnatal development (FIG. 23; B-E). However, with increased biotin concentration (e.g., 2 mg/ml), the BBB was susceptible to this high level of tracer until p18 (FIG. 24; I-K). Interestingly, this coincides with the timing of EBA expression.

### Example 2

Staining with SMI71 (anti-EBA antibody): Adult rats (Sprague Dawley) were anesthetized with a intra peritoneal injection of a ketamine/Xylazine cocktail. The thoracic cavity of the rats were dissected open exposing the heart. The right atrium of the heart was clipped with a fine scissors, and then phosphate buffered saline was perfused into the left ventricle of the heart for 10 minutes, followed by perfusion with 4% paraformaldehyde. Fixed brains were dissected, and further submersion fixed in 4% PFA overnight followed by equilibration in 30% sucrose for an additional night. The brain and peripheral tissue were then frozen in a 2:1 mixture of 30% sucrose:OCT and 10-20 micron sections were cut using a cryostat. Brain and tissue sections were blocked with methanol/0.3% hydrogen peroxide mixture for 30 minutes followed by 50% goat serum for an additional 30 minutes. SMI71 antibody (Covance) was then incubated overnight at 4 degrees.

Visualization of SMI71 was performed using a vectastain mouse IgG ABC kit (Vector labs PK6102) followed by DAB peroxidase substrate reaction (Vector labs SK-4100). For dissociated cultures, adult rats were euthanized with carbon dioxide. The cortical regions of the brains were dissected and enzymatically digested with 165 Units of papain containing L-cysteine (0.4 mg/ml) and DNAse (125 U/ml) for 30 minutes at 35 degrees. After mechanical separation by triturating through 5 ml pipettes, the cells were recovered by centrifugation at 1000 g and plated on coverslips coated with poly D-lysine in 24 well plates (Falcon). Cells were stained with 1:1000 SMI71 antibody at 4 degrees overnight using the Vector ABC HRP kit, followed by fixation with 4% PFA, blocking with 50% goat serum and then incubation with goat-anti mouse alexa 488 conjugated secondary. For co-labelling experiments, endothelial cells were also labeled with 1:500 anti-VWF (DAKO) primary antibody and goat-anti rabbit alexa 594. Sections where rehydrated with ethanol, cleared with xylene and viewed under light microscopy.

### Example 3

Expression Cloning: An adult rat brain cDNA library purchased from Biochain, was transformed into E. coli and spread on LB-Ampicillin plates such that roughly 2000 colonies per plate. Colonies from each plate were scraped together to form a pool of colonies. DNA was isolated using a Qiagen miniprep kits and transfected into COS-1 cells using Lipofectamine 2000. Cells grown on glass converslips were then incubated at 4 celsisue overnight with 1:1000 SMI71 antibody in DPBS, followed by fixation in cold 4% PFA for 10 minutes, blocking in 50% goat serum for 30 minutes and then incubation with a goat-anti mouse alexa 488 antibody for 1.5 hours at room temperature. Coverslips were then mounted on slides using vectashield with DAPI and visualized by fluorescence microscopy. DNA from the positive pool was then transformed into E. coli and plated at 200 colonies/dish.

Staining of COS-1 cells was performed using immunofluoresence as above. The positive pool was separated into pools of 200 using a sib selection technique. Briefly, the positive pool was transformed in E. coli, and then plated on LB-Amp with 200 colonies per plate. Individual plates were scraped to generate pools of 200. DNA was isolated and transfected into COS-1 cells as above. After identification of positive sub-pools, these were then narrowed down into pools of 20 followed by individual clones. Individual positive clones were sequenced by the Stanford PAN facility using T7 and M13 forward and reverse primers.

To test whether expression cloning would be a valuable technique for identification of EBA, specificity of the SMI71 antibody was determined. To accomplish this, live immunofluoresence staining was conducted on acutely dissociated adult rat cortical cells. In these cultures, the SMI71 antibody stained small rounded clumps of cells, that double labeled with the endothelial cell marker VWF (FIG. 24; F-G). The antibody did not stain other cells in this suspension suggesting that it was highly specific to the endothelial antigen. Because of the high degree of specificity of the SMI71 antibody, it was evident that any antigen generated from a adult rat brain cDNA library would be the EBA antigen. A molecular cloning approach was utilized comprising separating an adult rat brain cDNA expression library into pools of 1500-3000. After transfection of these pools into COS-1 cells, pools containing a positive clone were identified by live staining with the SMI71 antibody.

After screening over 300,000 clones, a single positive pool was identified (FIG. 25). Using a sib selection technique the number of clones in the pool was decreased until a single positive clone was obtained (FIG. 25F). After sequencing it was verified that this clone was Ngr2, a GPI linked molecule thought to be expressed in neurons.

Several approaches were used to confirm that the correct clone was identified. Ngr2 transfected COS-1 cells did not bind to various non-specific IgM control antibodies, or to the goat-anti mouse secondary antibodies utilized (FIG. 24), which indicates that the binding is specific to SMI71. Next, siRNA knockdown of Ngr2, and not off target molecules, inhibited binding of SMI71 to Ngr2 transfected COS-1 cells (FIG. 24; B, D, F). Furthermore, removal of GPI link through the enzymatic action of PI-PLC also disrupted SMI71 binding to Ngr2 transfected COS-1 cells (FIG. 24; E-F). Furthermore, the SMI71 antibody did not stain COS-1 cells transfected with two homologous proteins, Ngr and Ngr3 (FIGS. 24I and K). These experiments indicate that SM171 binds to Ngr2 in COS-1 cells, which demonstrate that the staining observed represents a specific interaction between the SMI71 antibody and the Ngr2 protein.

### Example 4

RT-PCR on purified Endothelial cells. Adult rats were euthanized with carbon dioxide. Brains and spleens were dissected out enzymatically dissociated as above. Endothelial cells were isolated by immunopanning, with anti-CD31 antibody (Research Diagnostics) after negative panning with a C5 antibody. After washing off unbound cells, the remaining endothelial cells were lysed with RLT buffer and the RNA was isolated using an RNAeasy kit from Qiagen. 35 cycles of RT-PCR was performed utilizing a number of primers identified on the medical genetics website (www2.eur.nl/fgg/chl/menu/menu2.html) taken from the rat Ngrh1 sequence (accession number AF532860; forward primers: cacagcgactcttcttgcag (SEQ ID NO: 1), actggacctcggtgacaatc (SEQ ID NO: 2), ttgcagaacaacctcattcg (SEQ ID NO: 3), ctcaccctgtggctcttctc (SEQ ID NO: 4). Reverse primers: gattgtcaccgaggtccagt (SEQ ID NO: 5), aggaaaaggtggctcaggtt (SEQ ID NO: 6), gattgtcaccgaggtccagt (SEQ ID NO: 7), tagtgactgcagcctctcca (SEQ ID NO: 8)). PCR products were separated on a 1% agarose gel containing ethidium bromide and visualized with UV light.RT-PCR was performed utilizing primers directed against different regions of the Ngr2 cDNA on mRNA isolated from purified brain and spleen endothelial cells. Interestingly, Ngr2 was expressed in both brain and spleen endothelial cells, however there was a splice variant that was specific to brain endothelial cells (FIG. 26A). After sequencing this variant it revealed a transcript with a 23 base pair insertion, resulting in an open reading frame shift and a premature stop codon (FIG. 26B). Interestingly, this protein is predicted to be secreted to the cell surface but not GPI linked. Coincident with this, the PI-PLC enzymatic cleavage did not disrupt SMI71 binding to endothelial cells in dissociated rat cortical cultures.

Brain homogenates and vessels fractions were analyzed by western blotimmunoassay to confirm the existence of the splice variant. A commercially available Ngr2 antibody (R&D systems) was utilized for the western blot assay, as it reacts with a band in Ngr2 transfected COS-1 cells but not mock transfected cells (FIG. 26C). Rat brain vessels were isolated and homogenized with a Dounce homogenizer in DPBS on ice and spun at 2000 g for 10 minutes 4 celsius. The pellet was resuspended in PBS and layered on top of a 15% dextran solution (MW 35 kD-45 kD) and centrifuged at 3500 g for 55 minutes. The vessel fraction in the pellet was then collected, washed with PBS, resuspended in RIPA buffer and stored at −80 celsius. Vessel and brain homogenates were analyzed by SDS-PAGE, utilizing 1:500 goat-anti Ngr2 antibody followed by donkey anti-goat HRP conjugated secondary antibody.

Interestingly, the brain homogenate contained the predicted full length Ngr2, while the vessel fraction contained a smaller doublet (FIG. 26C). This doublet migrates higher than the predicted truncated Ngr2, but may contain post-translational glycosylations.

The fact that SM171 is specific to endothelial cells, specific for exogenous Ngr2 expressed in COS-1 cells, and the fact that Ngr2 is expressed in CNS endothelial cells, suggest that Ngr2 is EBA.

### Example 5

Isolation of Vessel fraction: Adult rats were euthanized with carbon dioxide. Brains were dissected out and homogenized in cold PBS. After recovery by centrifugation at 1500 g, pellets were resupended in 0.5 M sucrose and layered over a 1.0-1.5 M sucrose gradient. After centrifugation, the vessel fraction pellet was recovered as well as the brain parenchyma interface. These samples were centrifuged and resuspended in RIPA buffer to be analyzed by western blot.

Western Blot: Samples were separated on 10% SDS-PAGE gels and transferred to PVDF membranes. After blocking in 5% milk for 1 hour at room temperature, membranes were incubated with 1:100 anti-Ngrh1 antibody (R&D systems) overnight at 4 degrees followed by secondary donkey anti-goat HRP conjugated secondary. Signals were developed using a Chemiluminescence Substrate system (Pierce) and exposed on X-ray film.

RT-PCR is used on purified endothelial cells to demonstrate not only that Ngrh1 is expressed in brain endothelial cells, but there is a splice form that is specific to brain endothelial cells and not expressed in other endothelial cells. Furthermore, by western blot on fractionated rat brains using a commercially available Ngrh1 antibody, it was demonstrated that there is a specific doublet band recognized by the Ngrh1 antibody present in the vessel fraction that is absent from the rest of the brain. Due to the remarkable specificity of the SMI71 antibody, and the presence of Ngrh1 in brain endothelial cells, it was verified that the correct target was identified.

We have observed that in brains sections, SMI71 antibody staining is restricted to blood vessels, while in dissociated cultures it specifically stains endothelial cells. Therefore, it follows that any protein product expressed from a rat brain cDNA library that bound to SMI71 would be the desired antigen. An adult rat brain cDNA expression library (biochain) was separated into pools of 2000-4000 clones, and utilized to transfect COS-1 cells with each pool. Using immunofluoresence microscopy, it was determined which cells bound SMI71. After analyzing 84 pools containing over 300,000 clones, one positive pool was identified. Using a sib selection technique the pool size was narrowed down until ingle positive clone was identified. This clone was not recognized by a number of isotype specific controls, suggesting that binding was specific to the SMI71 antibody. After sequencing the molecular target identified as Ngrh1.

RT-PCR on purified endothelial cells was utilized to demonstrate not only that Ngrh1 is expressed in brain endothelial cells, but that there is a splice variant form that is specific to brain endothlelial cells and not expressed in other endothelial cells. Furthermore, by western blot on fractionated rat brains using a commercially available Ngrh1 antibody, it is evident that there is a specific doublet band recognized by the Ngrh1 antibody present in the vessel fraction that is absent from the rest of the brain. Due to the remarkable specificity of the SMI71 antibody, and the presence of Ngrh1 in brain endothelial cells, it is clear that the correct target has been identified.

### Example 6

BBB in NgRH1 deficient mice. Interestingly, EBA is expressed late in BBB maturation, appearing in brain sections starting at about p15 or p17, and then throughout all CNS levels by p20.

BBB in Ngrh1 deficient mice are examined, both functionally by systemic delivery of tracers, as well as ultrastructurally by electron microscopy. Ngrh1 deficient mice are viable and indicating that they do not have large defects in the BBB, as this would be fatal (e.g., lethal hemorrhaging). Therefore, such mice may possess more subtle defects that can be detected by higher concentrations of tracers or through ultrastructural analysis. Such a case is observed in occludin deficient mice, which have normal permeability to tracers, but calcification of the brain suggests subtle changes in the BBB permeability to calcium.

To determine whether Ngrh1 deficient mice have functional deficits in the BBB, heterozygote mice are bred to generate Ngrh1 deficient mice and wild type and heterozygote litter mates. Adult mice are perfused with tracers of varying molecular weights including biotin (500D) as well as 10 kD and 70 kD tetramethyl rhodamine dextrans. The brains of these mice are fixed in 4% paraformaledehyde, sunk in 30% sucrose and embedded in OCT. After generating 10 micron cryosections of the mice brains, spinal cords and optic nerves, the tracers are visualized by fluorescent microscopy. The dextran tracers can be visualized directly, while the biotin tracers will visualized after staining with a streptavidin conjugated to alexa-488. We will also vary the concentration of the tracers. Biotin is utilized from 1.0-2.0 mg/ml while the dextrans are utilized at 0.5-2.0 mg/ml.

To assess the function of the BBB tracers are assayed to determine whether they are present within the lumen of the capillaries or diffuse throughout the brain parenchyma (i.e., increased permeability/barrier breakdown). Data obtained will provide whether the BBB is compromised to high concentrations of tracer (2.0 mg/ml biotin) but not low concentrations (1.0 mg/ml biotin). This sensitive assay will determine whether the BBB in these animals is fully mature or remains in an early developmental stage. If this is the case it suggests that Ngrh1 is required for the final step of BBB maturation, leading to the low permeability in adults. This would be very interesting as several studies suggest that small perturbations in the BBB may have large consequences on brain development and function. For example, polymorphisms in Claudin 5 have been linked to schizophrenia humans, while mutations in Moody, a protein required for the fly BBB, lead to altered sensitivity to drugs commonly abused.

### Example 7

BBB structure in NgRH1 deficient mice. Adult rodent brain endothelial cells are morphologically distinct from endothelial cells lacking barrier properties. Brain endothelial cells are held together by tight junctions, contain few endocytic vesicles, and lack fenestra in their cell membranes. Interestingly, systemic injection of the SMI71 antibody appears to affect each of these structures. Electron microscopy has demonstrated that the BBB of mice injected with this antibody have weaker tight junctions, more endocytic vesicles and appear to form fenestra. Indeed systemic injection of SMI71 breaks down BBB.

Rats were injected with either an anti-EBA antibody or an anti-CD31 antibody control that binds to live endothelial cells. After 20 minutes the rats were perfused with the biotin tracer, which was then visualized in frozen sections after paraformaldehyde fixation. The anti-EBA antibody was able to disrupt the BBB, allowing the tracer to leak into the brain parenchyma (FIG. 24A-B). This occurred in all regions of the brain including the cortex, cerebellum and optic nerve. On the other hand the CD31 antibody had no effect on the permeability of the CNS vessels. Interestingly, because perfusion fix is performed directly after the tracer perfusion, the specific segments of the vessels in which the segments are broken down were visualized (FIG. 24). Interestingly, at a dose of 40 ul SMI71 antibody/kg, only a small number proportion of the vasculature is permeable to the tracers.

Furthermore, the presence of tight junctions, fenestra and number of endocytic vesicles are compared between mutant and litter mates. If any ultrastructural defects are observed in adult Ngrh1 mutants, a comparison is made of mutant and wild type animals from early postnatal development (p2 and p8), a time prior to EBA expression. If differences are present in adults and not during development, then alternative mechanisms are involved in BBB maintenance. Phenotypic changes in the parenchyma of adult Ngrh1 mutants, including mineralization of the brain and degeneration of neural cells, indicate defects in the barrier to specific ions or molecules.

Tight junctions in Ngrh1 deficient mice are examined by freeze fracture electron microscopy. Freeze fracture is a technique used to visualize intermembrane junctions with great resolution. Capillary specimens isolated by dextran density centrifugation are fixed in glutaraldehyde, quick frozen in liquid nitrogen, fractured in a double replica device and analyzed by transmission electron microscopy. This technique allows visualization of the tight junction particles within extracellular (E-face) and the protoplasmic (P-face) sides of the membrane. The tight junction number can be quantified by counting the number of strands, and the complexity can be calculated using the ratio of branch points to length of the junctions. Brain endothelial cells have a characteristic tight junction morphology when analyzed by freeze fracture. They contain particles on both the P-face and the E-face membrane with a ratio of 55:45. This is in contrast to non-brain endothelial cells, in which particles are mostly on the E-face. Freeze fracture is utilized to compare the P-face:E-face particle ratio of Ngrh1 deficient mice with litter mate controls to elucidate whether there are small perturbations in the tight junctions of these mice.

The tight junction protein content of BBB tight junctions can also be assessed both by western blot and immunofluoresence microscopy. Brain endothelial cells express ZO-1, occludin, claudin 5 and claudin 12 at the tight junctions, for each of which commercially available antibodies are available. Brain capillaries from Ngrh1 deficient mice and litter mates controls can be isolated by density centrifugation and the protein levels of each tight junction protein measured by western blot. Furthermore, brains of each of mutant and littermates can be fixed and analyzed by immunofluoresence microscopy to determine if there is mislocalization of any of these proteins. These data will determine whether tight junction (TJ) strands are structurally intact, whether TJ expression/protein levels are normal and whether such proteins are localized to the TJ.

### Example 8

NgRH1 signaling and BBB permeability. By administering NgRH1 agonist and antagonist to a Ngrh1 deficient mice, wild type and heterozygote litter mates, after a series of neural insults, including EAE, stroke and nerve crush, differences in BBB permeability can be assayed. Due to the fact that the SMI71 antibody is able to transiently disrupt the BBB, modulating Ngrh1 can provide a way for administering therapeutics and/or diagnostics for a variety of CNS diseases.

Accordingly, one or more biologically active agents (therapeutic) of the present invention, as provided herein above, are administered to a subject. For example, antibodies targeting NgRH1 (or peptides, nucleic acid molecules or aptamers) are administered in an effective dose determined based on the animal subject and metrics known in the art for dosage, for example, weight of the animal, route of delivery, selected biologically active agent(s) or age, as also disclosed in U.S. Pat. No. 4,938,949. Therefore, once an animal is subjected to a neural insult, causing BBB leakage, the selected therapeutic is administered to restore the BBB (i.e., barrier integrity) by blocking Ngr2 signaling, thus ameliorating the disease or condition.

In addition, the same animals can be utilized to screen different NgRH1-specific candidate compounds (or agents) to determine whether such compounds are NgRH1 agonist or antagonist. In other words, where the candidate agent restores the BBB then it is determined that said compound is an agonist, while if the BBB permeability increases further, then the candidate agent is deemed to be an antagonist. Permeability can be measured by systemic administration of one or more enzymes, dyes, tracers or markers known in the art (collectively “tracers”).

### Example 9

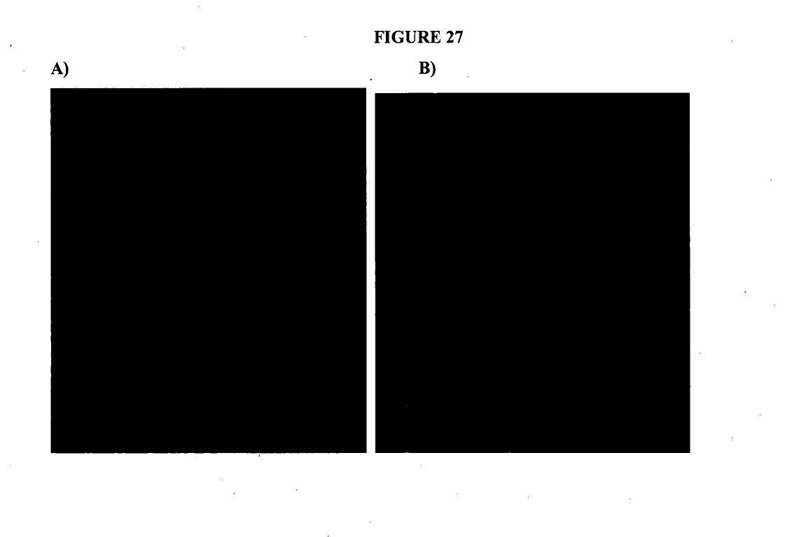

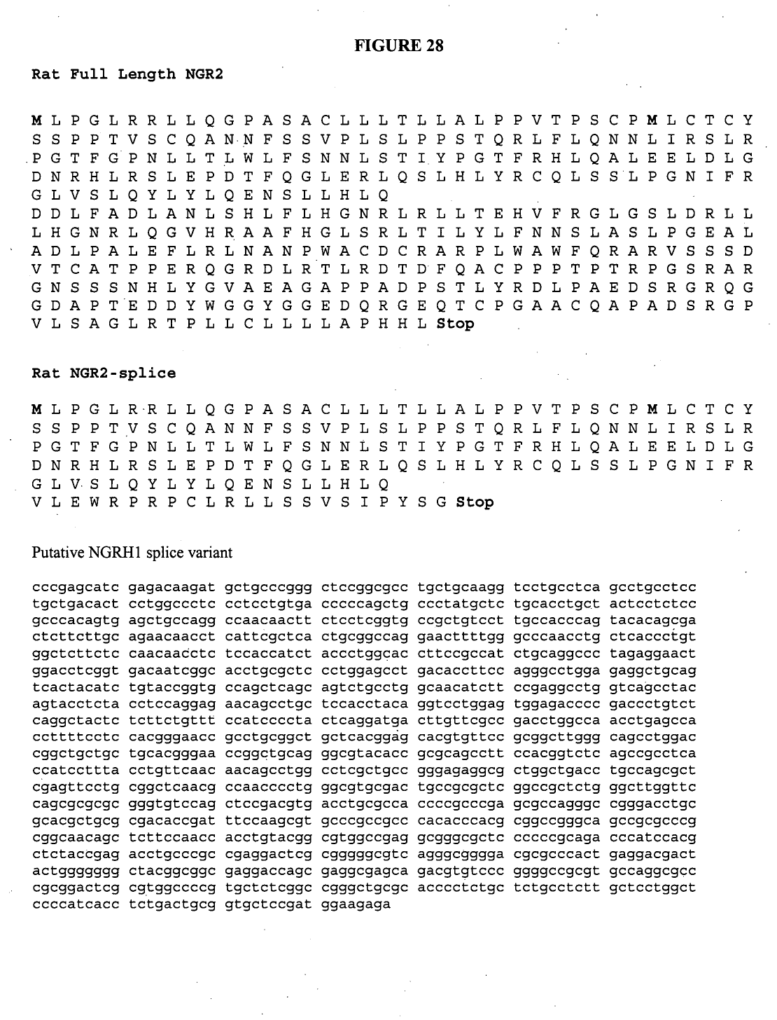

Systemic Injection of Ngr2 ligand disrupts the BBB. Ngr is known to be involved in growth cone collapse following binding of the ligands Nogo, OMgp and MAG, while signaling through the co-receptors p75, lingo and troy. To test whether binding Ngr2 to a ligand was sufficient to breakdown the BBB, the permeability of CNS vessels was analyzed after systemic injection of MAG. Interestingly, after injection of MAG, and not a control protein, the BBB was leaky to biotin (FIG. 27), thus demonstrating that activation of Ngr2 can lead to a breakdown of the BBB. MAG was injected at 0.625 mg/kg diluted in 100 ul saline into the tail vein of Sprague Dawley rats. Tracers studies were performed 15 minutes following injections.

### Example 10

BBB, NgRH1 and Multiple Sclerosis. Experimental allergic encephalomyelitis (EAE) is a mouse model for multiple sclerosis in which the rodent is immunized to specific myelin components. As with the human disease, there is a breakdown of the BBB that accompanies the autoimmune response. This breakdown is significant as it allows immune molecules and cells access into the parenchyma of the brain and spinal cord where they can damage the white matter.

EAE can be induced utilizing methods known in the art. To compare BBB permeability, tracers are administered to test, reference and/or control animals. Ngrh1 mice have been developed in a C57BL/6 background. In this strain, a MOG epitope (available from Princeton Biomolecules), is mixed with M. Tuberculosis H37A in IFA and injected s.c. into mice in conjunction with an i.p. injection of perussis toxin. This innocluation generates a chronic autoimmune disease in which severity can be monitored on a scale of 1-5 by strength of tail and limbs. In this experiment we will compare BBB breakdown in Ngrh1 deficient mice and littermate controls at various time points (e.g., selected number of days after inoculation).

BBB leakage can be quantified by cardiac perfusion of biotin followed by flushing the vessels with PBS. Biotin content in dissected brains., spinal cords and optic nerves are quantified by western blot using a strept avidin-HRP. BBB breakdown can also be analyzed qualitatively by tail vein injection of evans blue dye. After cardiac perfusion of PBS to remove dye from the vessels, brains, spinal cords and optic nerves will then be dissected and extent of evans blue leakage in the parenchyma is compared visually. Therefore, an agonist or antagonist of NgRH1, selected from the biologically active agents described herein above, is administered to the MS animal. Data obtained reflecting animals' tail and limb strength will determine the severity of disease. Therefore, if an agonist is administered then BBB permeability is restored thus ameliorating the induced MS model. Conversely, if an antagonist is administered then tail and limb strength will decrease further (disease state is exacerbated).

### Example 11

NgRH1 and Stroke. During a stroke, ischemia leads to a focal breakdown of the BBB. This disruption is thought to contribute to the neural damage and long term symptoms observed in patients. In this experiment we will determine whether Ngrh1 is necessary for the breakdown of the BBB following ischemia. To accomplish this Ngrh1 deficient and litter mate control mice are subjected to a middle cerebral artery occlusion for 1 hour by suture of this vessel. Breakdown of the BBB is quantified on occluded and control hemispheres at 1, 2, and 4 days post ischemia by cardiac perfusion of biotin. At these time points BBB disruption is also qualitatively analyzed by tail vein injection of evan's blue dye.

If Ngrh1 mutant mice show less disruption of the BBB following stroke, then the recovery from this injury in both mutant and wild type is determined. There are several standard tests which determine fine motor recovery following ischemia. In the rotor rod test the mouse is placed on a rotating cylindrical rod in which the speed is increased periodically. The length of time on the rod determines how the motor function of the mouse. In the open field activity test, the mouse is placed in a automated cage with infrared detection photocells. Animal movement, including length of path and number and duration of hind feet standing are recorded to monitor motor activity. These experiments will provide a measure of how much the BBB contributes to the pathology of stroke.

### Example 12

BBB and Nerve Injury. Following a crush injury to the peripheral nerve, the blood-nerve barrier (BNB) breaks down along the full length of the nerve distal to the crush site (e.g., following PNS injury), while the BBB only breaks down at the site of injury. This breakdown allows serum components and cells into the nerve to clear out myelin debris and pave the way for rapid regeneration. EBA is expressed by endothelial cells in the peripheral nerves and expression is lost during such breakdowns. Therefore, Ngrh1 signaling is required for the breakdown of the blood-nerve barrier after sciatic nerve crush. The difference in barrier breakdown in the CNS and PNS, may result from a difference in the clearance of myelin debris. Furthermore, if signaling through all Ngr family receptors is sufficient to breakdown of the BBB, then OMgp and Nogo may also be utilized in the same respects.

For example, agonist or antagonist are administered to blood-nerve barrier (BNB) of Ngrh1 deficient mice and littermates and permeability assessed in sciatic nerve distal to a crush injury. Crush injury is performed on the sciatic nerve and the permeability of the BNB is monitored 2-6 days after crush by perfusion of biotin and dextran tracers. In other work in the lab, we have determined that serum components are required for rapid removal of myelin and fast regeneration. If the BNB of Ngrh1 mice does not breakdown distal to a crush injury, then such mice will fail to complete Wallerian Degeneration (WD) and regenerate. WD in these mice is measured by western blot of distal segments of sciatic nerve for P0 and MBP, while regeneration is monitored by electrophysiology. Interestingly, Ngr and its homologs have been implicated as inhibitors of regeneration, through binding of myelin proteins Nogo and MAG. Therefore, NgRH1 mutants will exhibit more regeneration in the CNS. However, Ngrh1 agonist, through opening of the BNB, may aid in regeneration in the PNS, and therefore NgRH1 antagonist will impart less regeneration.

### Example 13

Endothelial Cell Culture Assay. Human brain microvascular endothelial cells (BMEC) can be propagated in RPMI 1640 medium supplemented with 20% heat-inactivated FBS (Omega Scientific Inc., Tarzana, Calif.), 2 mM L-glutamine, 1 mM MEM sodium pyruvate (GIBCO), 1×MEM nonessential amino acid solution (Sigma), and 1×MEM vitamin solution (Sigma). These cells, which have been shown to exhibit the hallmark characteristics of the endothelium of the blood-brain barrier, have been extensively used to examine how bacteria (Escherichia coli, group B Streptococcus and Streptococcus pneumoniae, and Citrobacter spp.), monocytes, viruses (human immunodeficiency virus), and fungi (Candida albicans) enter the brain. EA.hy926 cells, which are derived as a fusion of A549 cells with human umbilical vein endothelial cells (HUVEC) are frequently used as a model of systemic endothelial cells. Such cells can be grown in high-glucose (4.5 g/liter) Dulbecco's modified Eagle medium (GIBCO) supplemented with 10% heat-inactivated FBS and 1(x)hypoxanthine-thymine supplement (GIBCO). Both endothelial cell cultures can be plated and propagated in 25-cm₂ flasks (Sarstedt Inc., Newton, N.C.).

The confluent cells can be removed from the flasks using trypsin-EDTA solution and adjusted to 10₅/ml. Human BMEC and EA.hy926 cells were seeded (200 μl) on top of collagencoated semipermeable Transwell polycarbonate tissue culture inserts (6.5-mm diameter 0.33 cm, with a 3.0-μm pore size; Corning Costar Corp.). This in vitro model allows separate access to the upper compartment (blood side) and lower compartment (brain or tissue side). The cells can be cultured for 5 to 7 days in appropriate medium. Medium in both the top and bottom chambers is usually changed every other day. One day before the experiments, the culture medium is changed to experimental medium consisting of Ham's F-12 nutrient medium diluted 1:1 with medium M199 supplemented with 20% FBS and 2 mM Lglutamine (experimental medium) and incubated overnight. Electrical resistance measurements using an Endohm chamber with an EVOM voltometer (World Precision Instruments, Sarasota, Fla.) can be used to determine monolayer integrity and are expressed as ohms times centimeters squared, as per the manufacturer's recommendation, after adjustment for the resistance of the membrane itself.

Therefore, candidate NgRH1-specific agents obtained through one or more methods described herein (e.g., aptamers, antisense, proteins, peptides or antibodies), can be administered to the cell cultures to determine whether said candidate agents increase or decrease permeability, as measured through electrical resistance. As such, an increase in electrical resistance identifies an agonist while a decrease in electrical resistance identifies an agonist.

### Example 14

3-D Culture. In another example, isolated endothelial cells, pericytes and astrocytes (Endothelial-Pericytes-Astrocytes, or EPA) can be isolated utilizing one or more methods described herein, and subsequently cultured in suitable medium, supplemented with neurotropic and angiogenic growth factors. As such the EPA culture will provide a BBB model which can subsequently be utilized in BBB tracer assays, as well as in one or methods of the present invention in screening agonist and antagonist of barrier permeability.

Essentially the culture provides an EPA 3-dimensional cell culture system. These cultures will consist of cells mixed in a ratio of 1:1 and suspended in a collagen matrix, that solidifies at room temperature and is then overlaid with media. After 24 hours in a 3-dimensional culture, the endothelial cells aregin to rearrange and form tube-like structures, which the astrocyte end feet contact.

Additional matrices (e.g., matrigel, peptide scaffolds or fibronectin) known in the art can be utilized in producing the 3-D culture. The use of matrigel would be preferable as it is more cost effective. Differentiation of the cells and barrier phenotype will also be confirmed by fluorescent microscopy and/or western blotting, this will enable morphological analysis and accurate localization of the cells within the culture.

In addition, one or more cell types can be transfected to express a desired gene (e.g., endothelial cells to provide altered expression levels of NgRH1). In this regard, expression of the desired gene can be regulated through inducible promoters known in the art (e.g., tet-responsive), or as described herein above. Supra, Table 1. Furthermore, changes in gene and protein expression as a result of contact with the other cell types, can be identified and monitored, using molecular biology techniques, FACS analysis sorting on known cell-specific markers, as well as ELISA and RIA techniques to measure the release of different cytokines and growth factors such as VEGF and Epo into the culture media. These methods will allow identification of proteins expressed in the BBB under normal physiological conditions.

Moreover, the cell culture assay can provide an efficient means for assaying analyzing the effects form various gene expression profiles, in a constitutively or temporally regulated context, to determine what effects a particular gene product imparts on BBB permeability. Permeability can be assessed utilizing fluorescence staining techniques described herein above or as practiced in fluorescence detection techniques known in the relevant art.

Alternatively, or in addition to transfecting one or more cell types, the EPA culture can also be utilized to screen various biologically active agents to determine their effects on BBB premeability. For example, chemical compounds, proteins, peptides, antibodies, antisense or siRNA targeting various cell surface receptors (including NgRH1) can be screened in the EPA culture to determine what additional factors are involved in the NgRH1 signaling mechanisms that regulate BBB permeability.

### Example 15

Purification of Endothelial Cells: Optic nerves were dissected from 1-2 litters of embryonic or postnatal Sprague dawley rats. A single cell suspension was made through enzymatic dissociation with 40 units of papain for 20 minutes followed by mechanical separation by triturating with 21 and 23 gauge needles. The cells were incubated on a dish coated with C5 antibody to deplete astrocyte lineage, oligodendrocyte lineage cells and microglia. The supernatant from these dishes was then incubated on Petri dish coated with an antibody directed against CD31 to bind endothelial cells. Cells from the supernatant were washed off and endothelial cells were recovered through trypsinization. Endothelial cells were cultured in NB-SATO containing bFGF on coverslips coated with collagen IV.

Purification of Pericytes: Optic nerves were dissected from 1-2 litters of postnatal day 7 sprague dawley rats. A single cell suspension was made through enzymatic dissociation with 85 units of papain for 45 minutes followed by mechanical separation by triturating with 21 and 23 gauge needles. The cells were allowed 30 minutes at 37 degrees to recover lost epitopes and then were incubated on a dish coated with C5 antibody to deplete astrocyte lineage, oligodendrocyte lineage cells and microglia. The supernatant from these dishes was then incubated on Petri dish coated with an antibody directed against PDGFRbeta to bind pericytes. Cells from the supernatant were washed off and pericytes were recovered through trypsinization. The pericytes were then cultured in DMEM containing 110% FCS, penicillin, streptomycin, glutamine, insulin and pyruvate.

### Example 16

Transplantation of purified cells: Purified cells were centrifuged at 1000 rpm for 10 minutes. Cell pellets were then resuspended at 100,000 cells/ul in DPBS. Adult Sprague dawley rats were anesthetized using ketamine/xylazine cocktail. With a 30 gauge needle, a small hole was made in the anterior chamber of the eyeball. A similar hole was made in the posterior chamber to equalize the pressure. Utilizing a wiretool micropipette, 5-10 ul of cells were injected into the anterior chamber. Rats were allowed 2 weeks for recovery before analysis.

What is claimed is:

1. A method of modulating blood-brain barrier (BBB) permeability comprising administering an agent to a subject, wherein said agent targets a human Nogo receptor 2 (NgR2) that is present in the brain.

2. A method of modulating blood-brain barrier (BBB) permeability of a subject comprising administering a ligand of NgR2 to said subject.

3. The method of claim 1 or 2, wherein the agent increases BBB permeability.

4. The method of claim 1 or 2, wherein the agent decreases BBB permeability.

5. The method of claim 1 or 2, wherein said modulating is reversible.

6. The method of claim 1 or 2, where said agent is selected from the group consisting of inorganic molecule, peptide, peptide mimetic, antibody, liposome, small interfering RNA, antisense, aptamer, and external guide sequence.

7. A method of delivering a therapeutic agent to a central nervous system (CNS) of a subject comprising administering the therapeutic agent to the CNS prior to, concurrent with, or subsequent to, increasing BBB permeability as a result of modulating NgR2 activity and/or expression level.

8. The method of claim 7, wherein said NgR2 is human NgR2.

9. The method of claim 8, wherein modulating NgRH1 cell surface protein activity and/or expression level is effected by an exogenous agent that targets NgR2.

10. The method of claim 9, where said agent is selected from the group consisting of inorganic molecule, peptide, peptide mimetic, antibody, liposome, small interfering RNA, antisense, aptamer, and external guide sequence.

11. The method of claim 7, wherein the increase in BBB permeability is transient.

12. The method of claim 7, wherein the agent is a ligand of NgR2.

13. A method of assessing whether a candidate agent modulates BBB permeability comprising the steps of:
a. exposing a subject's central nervous system to an indicator of said BBB permeability;
b. administering to said subject said candidate agent that targets NgR2, wherein an increases or decrease in BBB permeability indicates that said candidate agent is capable of modulating BBB permeability.

14. The method of claim 13 where said agent is an agonist of NgR2.

15. The method of claim 13 where said agent is an antagonist of NgR2.

16. The method of claim 13 where NgR2 is human NgR2.

17. The method of claim 13 where said agent is selected from the group consisting of inorganic molecule, peptide, peptide mimetic, antibody, liposome, small interfering RNA, antisense, aptamer, and external guide sequence.

18. The method of claim 13, wherein said subject is a transgenic animal that is NgR2 deficient.


SEQUENCE LISTING (14 sequences)

SEQ ID NO: 1 - DNA, 20 bp, Artificial Sequence
  Description of Artificial Sequence Synthetic primer
cacagcgactcttcttgcag

SEQ ID NO: 2 - DNA, 20 bp, Artificial Sequence
  Description of Artificial Sequence Synthetic primer
actggacctcggtgacaatc

SEQ ID NO: 3 - DNA, 20 bp, Artificial Sequence
  Description of Artificial Sequence Synthetic primer
ttgcagaacaacctcattcg

SEQ ID NO: 4 - DNA, 20 bp, Artificial Sequence
  Description of Artificial Sequence Synthetic primer
ctcaccctgtggctcttctc

SEQ ID NO: 5 - DNA, 20 bp, Artificial Sequence
  Description of Artificial Sequence Synthetic primer
gattgtcaccgaggtccagt

SEQ ID NO: 6 - DNA, 20 bp, Artificial Sequence
  Description of Artificial Sequence Synthetic primer
aggaaaaggtggctcaggtt

SEQ ID NO: 7 - DNA, 20 bp, Artificial Sequence
  Description of Artificial Sequence Synthetic primer
gattgtcaccgaggtccagt

SEQ ID NO: 8 - DNA, 20 bp, Artificial Sequence
  Description of Artificial Sequence Synthetic primer
tagtgactgcagcctctcca

SEQ ID NO: 9 - DNA, 1304 bp, Rattus norvegicus
cccgagcatcgagacaagatgctgcccgggctccggcgcctgctgcaaggtcctgcctcagcctgcctcctgctgacactcctggccctccctcctgtgacccccagctgccctatgctctgcacctgctactcctctccgcccacagtgagctgccaggccaacaacttctcctcggtgccgctgtccttgccacccagtacacagcgactcttcttgcagaacaacctcattcgctcactgcggccaggaacttttgggcccaacctgctcaccctgtggctcttctccaacaacctctccaccatctaccctggcaccttccgccatctgcaggccctagaggaactggacctcggtgacaatcggcacctgcgctccctggagcctgacaccttccagggcctggagaggctgcagtcactacatctgtaccggtgccagctcagcagtctgcctggcaacatcttccgaggcctggtcagcctacagtacctctacctccaggagaacagcctgctccacctacaggatgacttgttcgccgacctggccaacctgagccaccttttcctccacgggaaccgcctgcggctgctcacggagcacgtgttccgcggcttgggcagcctggaccggctgctgctgcacgggaaccggctgcagggcgtacaccgcgcagccttccacggtctcagccgcctcaccatcctttacctgttcaacaacagcctggcctcgctgccgggagaggcgctggctgacctgccagcgctcgagttcctgcggctcaacgccaacccctgggcgtgcgactgccgcgctcggccgctctgggcttggttccagcgcgcgcgggtgtccagctccgacgtgacctgcgccaccccgcccgagcgccagggccgggacctgcgcacgctgcgcgacaccgatttccaagcgtgcccgccgcccacacccacgcggccgggcagccgcgcccgcggcaacagctcttccaaccacctgtacggcgtggccgaggcgggcgctccccccgcagacccatccacgctctaccgagacctgcccgccgaggactcgcgggggcgtcagggcggggacgcgcccactgaggacgactactgggggggctacggcggcgaggaccagcgaggcgagcagacgtgtcccggggccgcgtgccaggcgcccgcggactcgcgtggccccgtgctctcggccgggctgcgcacccctctgctctgcctcttgctcctggctccccatcacctctgactgcggtgctccgatggaagaga

SEQ ID NO: 10 - DNA, 1263 bp, Mus musculus
atgctgcccgggctccggcgcctgctgcaaggtcctgcctcagcctgcctactgctgacactcctggcccttccttccgtgacccccagctgtcctatgctctgcacctgctactcctccccgcccaccgtgagctgccaggccaacaacttctcctcagtgccgctgtccttgccacccagtacacagagactcttcttgcagaacaacctcatccgctcactgcggccaggcacctttgggcccaacctgctcaccctgtggctcttctccaacaacctctccaccatccaccctggcaccttccgccacctgcaggccctagaagaactggacctcggtgacaaccggcacctgcgctccctggagcccgacaccttccagggtctggagaggctgcagtcactacacctgtatcgttgccagctcagcagcctgcctggcaacattttccgaggcttggtcagcctacagtacctctacctccaggagaacagcctgctccatctacaggatgacttgttcgcggacctggccaacctgagccacctcttcctccacgggaaccgcctgcggctgctcacggagcacgtgttccgcggcttgggcagcctggaccggctgttgctgcacgggaaccggctgcagggcgtgcaccgcgcggctttccacggcctcagccgcctcaccatcctctacctgttcaacaacagcctggcctcgctgccgggagaggcgctggccgacctgccggcgctcgagttcctgcggctcaacgccaacccctgggcgtgcgactgccgcgctcggccgctctgggcttggttccagcgcgcgcgggtgtccagctccgacgtgacctgcgccaccccgcccgagcgccagggccgggacctgcgcgcgctgcgcgactccgatttccaagcgtgcccgccgcccacgcccacgcggccgggcagccgcgcccgcggcaacagctcttccaaccacctgtacggcgtggccgaggctggcgctccccccgcagacccgtccacgctctaccgagatctgcccgccgaggactcgcgggggcgccagggcggggacgcgcccaccgaggacgactactgggggggctacggcggcgaggatcagcggggcgagcagacgtgtcccggggccgcgtgccaggcgcccgcagactcgcgtggccccgcgctctcggccgggctgcgcacccctctgctctgcctcttgcccctggcgctccatcacctctga

SEQ ID NO: 11 - DNA, 1263 bp, Homo sapiens
atgctgcccgggctcaggcgcctgctgcaagctcccgcctcggcctgcctcctgctgatgctcctggccctgcccctggcggcccccagctgccccatgctctgcacctgctactcatccccgcccaccgtgagctgccaggccaacaacttctcctctgtgccgctgtccctgccacccagcactcagcgactcttcctgcagaacaacctcatccgcacgctgcggccaggcacctttgggtccaacctgctcaccctgtggctcttctccaacaacctctccaccatctacccgggcactttccgccacttgcaagccctggaggagctggacctcggtgacaaccggcacctgcgctcgctggagcccgacaccttccagggcctggagcggctgcagtcgctgcatttgtaccgctgccagctcagcagcctgcccggcaacatcttccgaggcctggtcagcctgcagtacctctacctccaggagaacagcctgctccacctacaggatgacttgttcgcggacctggccaacctgagccacctcttcctccacgggaaccgcctgcggctgctcacagagcacgtgtttcgcggcctgggcagcctggaccggctgctgctgcacgggaaccggctgcagggcgtgcaccgcgcggccttccgcggcctcagccgcctcaccatcctctacctgttcaacaacagcctggcctcgctgcccggcgaggcgctcgccgacctgccctcgctcgagttcctgcggctcaacgctaacccctgggcgtgcgactgccgcgcgcggccgctctgggcctggttccagcgcgcgcgcgtgtccagctccgacgtgacctgcgccacccccccggagcgccagggccgagacctgcgcgcgctccgcgaggccgacttccaggcgtgtccgcccgcggcacccacgcggccgggcagccgcgcccgcggcaacagctcctccaaccacctgtacggggtggccgaggccggggcgcccccagccgatccctccaccctctaccgagatctgcctgccgaagactcgcgggggcgccagggcggggacgcgcctactgaggacgactactgggggggctacgggggtgaggaccagcgaggggagcagatgtgccccggcgctgcctgccaggcgcccccggactcccgaggccctgcgctctcggccgggctccccagccctctgctttgcctcctgctcctggtgccccaccacctctga

SEQ ID NO: 12 - protein, 420 aa, Rattus norvegicus
MLPGLRRLLQGPASACLLLTLLALPPVTPSCPMLCTCYSSPPTVSCQANNFSSVPLSLPPSTQRLFLQNNLIRSLRPGTFGPNLLTLWLFSNNLSTIYPGTFRHLQALEELDLGDNRHLRSLEPDTFQGLERLQSLHLYRCQLSSLPGNIFRGLVSLQYLYLQENSLLHLQDDLFADLANLSHLFLHGNRLRLLTEHVFRGLGSLDRLLLHGNRLQGVHRAAFHGLSRLTILYLFNNSLASLPGEALADLPALEFLRLNANPWACDCRARPLWAWFQRARVSSSDVTCATPPERQGRDLRTLRDTDFQACPPPTPTRPGSRARGNSSSNHLYGVAEAGAPPADPSTLYRDLPAEDSRGRQGGDAPTEDDYWGGYGGEDQRGEQTCPGAACQAPADSRGPVLSAGLRTPLLCLLLLAPHHL

SEQ ID NO: 13 - protein, 193 aa, Rattus norvegicus
MLPGLRRLLQGPASACLLLTLLALPPVTPSCPMLCTCYSSPPTVSCQANNFSSVPLSLPPSTQRLFLQNNLIRSLRPGTFGPNLLTLWLFSNNLSTIYPGTFRHLQALEELDLGDNRHLRSLEPDTFQGLERLQSLHLYRCQLSSLPGNIFRGLVSLQYLYLQENSLLHLQVLEWRPRPCLRLLSSVSIPYSG

SEQ ID NO: 14 - DNA, 1368 bp, Artificial Sequence
  Description of Artificial Sequence Synthetic polynucleotide sequence
cccgagcatcgagacaagatgctgcccgggctccggcgcctgctgcaaggtcctgcctcagcctgcctcctgctgacactcctggccctccctcctgtgacccccagctgccctatgctctgcacctgctactcctctccgcccacagtgagctgccaggccaacaacttctcctcggtgccgctgtccttgccacccagtacacagcgactcttcttgcagaacaacctcattcgctcactgcggccaggaacttttgggcccaacctgctcaccctgtggctcttctccaacaacctctccaccatctaccctggcaccttccgccatctgcaggccctagaggaactggacctcggtgacaatcggcacctgcgctccctggagcctgacaccttccagggcctggagaggctgcagtcactacatctgtaccggtgccagctcagcagtctgcctggcaacatcttccgaggcctggtcagcctacagtacctctacctccaggagaacagcctgctccacctacaggtcctggagtggagaccccgaccctgtctcaggctactctcttctgtttccatcccctactcaggatgacttgttcgccgacctggccaacctgagccaccttttcctccacgggaaccgcctgcggctgctcacggagcacgtgttccgcggcttgggcagcctggaccggctgctgctgcacgggaaccggctgcagggcgtacaccgcgcagccttccacggtctcagccgcctcaccatcctttacctgttcaacaacagcctggcctcgctgccgggagaggcgctggctgacctgccagcgctcgagttcctgcggctcaacgccaacccctgggcgtgcgactgccgcgctcggccgctctgggcttggttccagcgcgcgcgggtgtccagctccgacgtgacctgcgccaccccgcccgagcgccagggccgggacctgcgcacgctgcgcgacaccgatttccaagcgtgcccgccgcccacacccacgcggccgggcagccgcgcccgcggcaacagctcttccaaccacctgtacggcgtggccgaggcgggcgctccccccgcagacccatccacgctctaccgagacctgcccgccgaggactcgcgggggcgtcagggcggggacgcgcccactgaggacgactactgggggggctacggcggcgaggaccagcgaggcgagcagacgtgtcccggggccgcgtgccaggcgcccgcggactcgcgtggccccgtgctctcggccgggctgcgcacccctctgctctgcctcttgctcctggctccccatcacctctgactgcggtgctccgatggaagaga